(19) United States
(12) Patent Application Publication
(10) Pub. No.: US 2025/0019352 A1
(43) Pub. Date: Jan. 16, 2025

(54) PHARMACOLOGICAL AGENTS FOR TREATING OPHTHALMIC DISEASES

(71) Applicant: Plex Pharmaceuticals, Inc., San Diego, CA (US)

(72) Inventors: Santosh C. SINHA, Ladera Ranch, CA (US); Sridhar Govinda PRASAD, San Diego, CA (US); Albert REMACLE, Spring Valley, CA (US); Boris RATNIKOV, San Diego, CA (US)

(21) Appl. No.: 18/575,231

(22) PCT Filed: Jun. 29, 2022
(86) PCT No.: PCT/US2022/035558

§ 371 Date: Dec. 28, 2023

Related U.S. Application Data
(60) Provisional application No. 63/216,429, filed on Jun. 29, 2021.

(51) Int. Cl. C07D 285/36; A61K 31/381; A61K 31/426; A61K 31/554; A61P 27/12; C07C 323/65

(52) U.S. Cl. CPC C07D 285/36; A61K 31/381; A61K 31/426; A61K 31/554; A61P 27/12; C07C 323/65

(57) ABSTRACT

Methods for treating ophthalmic diseases and disorders such as treating presbyopia or cataract in a subject in need thereof. The methods require administering to the subject an effective amount of a composition comprising a compound that inhibits the formation of high molecular weight aggregates of human α-A-crystallin and/or human α-B-crystallin.

### CROSS-REFERENCE TO RELATED APPLICATIONS

This application claims the benefit of U.S. Provisional Application Ser. No. 63/216,429, which was filed on Jun. 29, 2021, and the entire contents of which is expressly incorporated by reference herein.

### FIELD OF THE DISCLOSURE

The disclosure relates to compounds and methods of treating ophthalmic diseases and disorders.

### BACKGROUND

Cataracts are the leading cause of blindness worldwide according to the World Health Organization (WHO), particularly in low- and middle-income countries. Data dating back to the beginning of this millennium shows that 30-60% of blindness in Africa and 60-80% in South East Asia is attributable to cataracts. In the United States, the current number of those with cataracts is estimated to be more than 25.7 million. Projections from Prevent Blindness research estimate that the number will increase to 38.5 million by 2032, and to 45.6 million by the year 2050. Cataract is a clouding of the eye's lens which blocks or changes the passage of light into the eye. Cataract usually forms in both eyes, but not at the same rate. It can develop slowly or quickly, or progress to a certain point, then not get any worse. Besides aging, other factors may cause cataracts to form. Eye infections, certain medicines (e.g., steroids), injuries, and exposure to intense heat or radiation may cause cataracts. Too much exposure to non-visible sunlight (called UV or ultraviolet light) and various diseases, such as diabetes or metabolic disorders, may also contribute to formation of cataracts.

The only cataracts treatment currently available is surgical extraction and replacement of eye lens with intraocular lens. This treatment represents a high public health burden. Although cataracts surgery is generally considered to be safe, there are significant complications: (i) 30-50% of patients in the US who have undergone cataracts surgery develop opacification of the posterior lens capsule within two years and require laser treatment; (ii) 0.8% have retinal detachments; (iii) 0.6-1.3% are hospitalized for corneal edema or require corneal transplantation and (iv) about 1% develop endophthalmitis. In addition, in many remote and poor areas of the developing and under-developed regions of the world, people still remain blind from cataract, primarily due to lack of access to eye care.

Presbyopia is the loss of accommodative ability of the eye resulting in the inability to focus on near objects. Presbyopia affects everyone over the age of 45 and has significant negative impacts on the quality of life. Current treatments for presbyopia include: (i) non-invasive approaches that utilize devices to help improve near and distance vision but do nothing to restore the natural process of accommodation and require constant use of the devices, and (ii) invasive surgical procedures which are associated with major complications including decrease in vision quality, regression effects, anisometropia, corneal ectasia, and haze. Most importantly, none of these methods can reverse presbyopia. Moreover, no treatment option exists that can either prevent or delay the onset of presbyopia.

Stiffening of eye lens and changes in the elasticity of the lens capsule, dimension of eye lens, dimension of the zonular attachment, and ciliary muscle (CM) contractions, have all been proposed as contributing factors for presbyopia. However, human and non-human primate studies suggest that CM function is normal well beyond the onset of presbyopia. By contrast, the human lens increases in stiffness with age in a manner that directly correlates with a loss in accommodative power [FIGS. 1A-1D]. The loss in accommodative power can be restored by implanting intraocular lenses made from a flexible polymer, suggesting that restoration of lens flexibility is sufficient to restore accommodation. Therefore, pharmacological agents that prevent or reverse the hardening of the crystalline lens hold promise for novel non-invasive treatment of presbyopia.

At the molecular level, proteins known as crystallins play a major role in the stiffening of the eye lens. The lens crystallins comprise three isoforms, α, β, and γ and make up 90% of the eye lens protein content. αcrystalline (AC), an ATP-independent chaperone and member of the small heat shock protein (sHsp) family, constitutes 40% of the crystallin protein content. It exists as a hetero-oligomer of two subunits, αA-crystallin (AAC) and αB-crystallin (ABC) and its expression is primarily restricted to the eye lens. It recognizes exposed conformational features in partially unfolded lens proteins and sequesters them from one another, thereby reducing the population of aggregation-prone species that would otherwise lead to various age-related vision impairment.

Multiple studies have established a link between stiffening of the human lens and AC function. Dynamic mechanical analysis measurements have shown that there is a significant increase in the stiffness of the lens with age, particularly in the lens nucleus where a 500- to 1000-fold decrease in elasticity is observed. This increase in lens stiffness correlates with the age-related decline in free AC chaperone concentration as most AC becomes incorporated into high molecular weight (HMW) aggregates by the age of 40-50. This conversion of soluble AC into HMW aggregates is accompanied by a large increase in lens stiffness, presumably because the low level of soluble AC present is not sufficient to chaperone denatured proteins. That age-related decrease in free AC chaperone is responsible for lens stiffness is supported by experiments where human lenses were subjected to heating to mimic the age-related conversion of soluble AC into HMW aggregates and an increase in lens stiffness was observed. Similarly, purified soluble AC forms HMW aggregates when exposed to UV radiation with a loss in chaperone like activity. The HMW aggregate is formed due to the intermolecular cross-linking, particularly S—S bonds, resulting from the oxidation of cysteine sulfhydryl groups (—SH). The formation of this disulfide cross-linked HMW aggregate is thought to be a major contributor in increasing the stiffness and loss of accommodation amplitude of the lens.

It has been suggested that presbyopia is the earliest observable symptom of age-related nuclear (ARN) cataract, a major cause of blindness in the world.

Given the need for noninvasive treatment that can protect and restore the accommodative ability of the eye lost in presbyopia and given that formation of HMW AC aggregates is a major causative factor underlying presbyopia, there is a need for the development of pharmacological agents that can selectively delay and/or reverse the HMW AC aggregate formation.

### BRIEF DESCRIPTION OF THE FIGURES

FIG. 1A: Stiffness values of cortex and nucleus as a function of lens age. Nuclear and cortical modulus values plotted on a log scale as a function of age for individual lenses. Nuclear values are shown in blue and cortical values in red. The curves were fitted using SigmaPlot. This figure was adapted from Heys K R, et. al., Molecular Vision 2004; 10:956-963.

FIG. 1B: Change in accommodative power (diopters) as function of age. Accommodative power of human subjects expressed as a function of age. The data points on the graph represent a summary of four separate studies on human accommodation as indicated by the following references. 1 (Blue): Bruckner, R., et. al., Ophthalmo-gerontological research results. Doc. Ophthalmol 1986; 64:235-310. 2 (Red): Duane, A J., J. Am Med Assoc. 1912; 59:1010-1013. 3 (Green): Donders F C., New Sydenham; 1864 and 4 (Open circle): Hamasaki, D., et., al., Am J Optom Arch Am Acad Optom 1965; 33: 3-14. This figure was adapted from Heys K R, et. al., Molecular Vision 2004; 10:956-963.

FIG. 1C: Mass distribution in the water-soluble fraction in young (19 years) (solid line and filled circle) and aged (83 years) (broken line andopen circle) human lenses using multiangle light scattering. This figure was adapted from Santhoshkumar P., et. al., THE JOURNAL OF BIOLOGICAL CHEMISTRY (2008) 283, NO. 13: 8477-8485.

FIG. 1D: Changes in the content of soluble α-crystallin (ο) and high molecular weight protein (●) in the lens nucleus as a function of age. This figure was adapted from Heys K R., et., al., Aging Cell (2007) 6:807-815.

FIG. 2A: SDS-PAGE showing the protection against UVC induced aggregation of human α-A-crystallin (hAAC) by disulfide and related SDMs. Shown are also efficacy of non-related SDMs. The compounds were tested at 500 μM concentration.

FIG. 2B: Molecular weight and structures of the disulfide SDMs and related compounds tested for protection against UVC induced aggregation of hAAC.

FIG. 3A: Molecular structures of the disulfide SDMs evaluated for protection of human lens epithelial cells, SRA 01/04 against UVC and heat.

FIG. 3B: Protection rendered by disulfide SDMs towards human lens epithelial cells, SRA 01/04 upon exposure to UVC (for 48 Secs). SRA 01/04 cells were pre-incubated for 2 hours with disulfide containing SDMs then exposed to UVC. Cell viability was assessed by Alamar blue 24-hours after exposure. Graphed are mean±SD.

FIG. 3C: Protection rendered by disulfide SDMs towards human lens epithelial cells, SRA 01/04 upon exposure to Heat (50° C. for 30 mins). SRA 01/04 cells were pre-incubated for 2 hours with disulfide containing SDMs then exposed to UVC. Cell viability was assessed by Alamar blue 24-hours after exposure. Graphed are mean±SD.

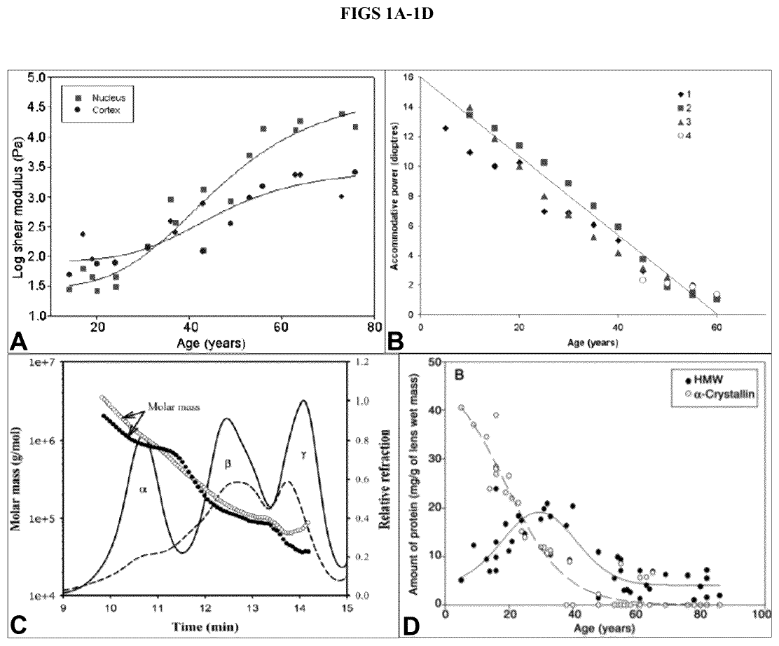

### SUMMARY OF VARIOUS ASPECTS

Small molecules that can inhibit the formation of high molecular weight (HMW) aggregates of human αA-crystallin (hAAC) are disclosed. These small molecules are called disaggregates (SMDs). It is believed that these SMDs would be useful in the treatment of presbyopia and also in the treatment of cataract including cataract that is age-related (nuclear sclerotic, cortical, and posterior subcapsular), congenital, secondary, traumatic, or caused by radiation.

Accordingly, compounds having the formula (I), (II), (III), (IV), or (V) shown below, and pharmaceutically acceptable salt thereof, method of making these compounds, and methods of using these compounds are disclosed. In some aspects, methods of treating eye-related conditions such as cataract or presbyopia using SMDs are provided. In some aspects, methods comprise administering an effective amount of a compound having the formula (I), (II), (III), (IV), or (V) shown below, or a pharmaceutically acceptable salt thereof, to a subject in need thereof, are provided. In some aspects, pharmaceutical compositions comprising a compound having the formula (I), (II), (III), (IV), or (V) shown below, or a pharmaceutically acceptable salt thereof, and one or more pharmaceutically acceptable excipients, are provided. In some aspects, the methods of administration disclosed herein comprise administration of the compounds having the formula (I), (II), (III), (IV), or (V), or administration of prodrug compounds that convert to these compounds in the body.

Compounds having formula (I), (II), (III), (IV), and (V), and prodrugs thereof are provided.

A compound of formula (I) is provided:

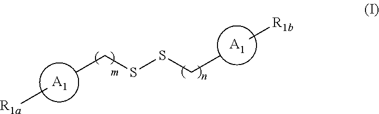

wherein R₁ₐ, R_{1b}, A₁, A₂, m, and n are as described herein below.

A compound of formula (II) is provided:

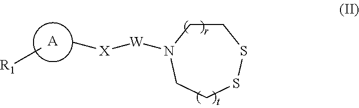

wherein R₁, A, X, W, r, and t, are as described herein below.

A compound of formula (III) is provided:

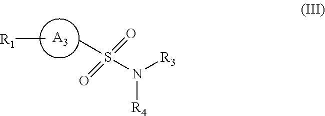

wherein R₁, R₃, R₄, and A₃ are as described herein below.

A compound of formula (IV) is provided:

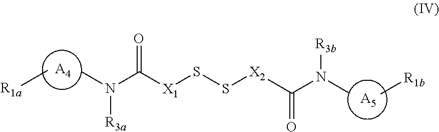

wherein R₁ₐ, R_{1b}, R₃ₐ, R_{3b}, X₁, X₂, A₄, and A₅ are as described herein below.

A compound of formula (V) is provided:

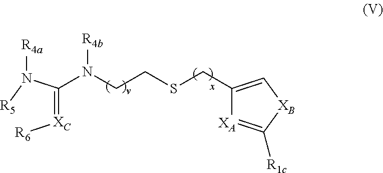

wherein R_{1c}, R₄ₐ, R_{4b}, R₅, R₆, X_{A}, X_{B}, X_{C}, v, and x, are as described herein below.

### DETAILED DESCRIPTION OF VARIOUS ASPECTS

### General Description of Compounds

In a first aspect, the present disclosure provides a compound of formula (I), and a method of use of these compounds. In one aspect, the present disclosure provides a method of treating, preventing, reducing the severity of, or reducing or alleviating the symptoms associated with, an eye-related disease or condition, including but not limited to cataract or presbyopia, in a subject in need thereof, the method comprising administering to the subject an effective amount of a composition comprising a compound having the formula (I):

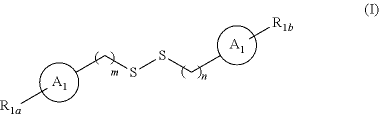

or a pharmaceutically acceptable salt thereof,
   wherein
A₁ and A₂ are, each independently, a six-membered aromatic or heteroaromatic ring comprising at least one heteroatom and at least one carbon atom;
each of m and n is, independently, any number from 0 to 4;
R₁ₐ is attached to one or more carbon atoms of A₁; and R_{1b} is attached to one or more carbon atoms of A₂;
R₁ₐ and R_{1b} are, each independently, selected from the group consisting of: hydrogen; (C₁-C₆)alkyl; halo(C₁-C₆)alkyl; (C₁-C₆)alkoxy; halo(C₁-C₆)alkoxy; hydroxy(C₁-C₆)alkyl; (C₁-C₆)alkoxy(C₁-C₆)alkyl; (C₂-C₅)alkenyl; (C₂-C₅)alkynyl; SO₂R₂; CO₂R₂; NH₂; (R₂)(R₂) wherein each R₂ may be the same or different, (C₁-C₆)alkylamine; (C₁-C₆)dialkylamine; NHCOR₂; hydroxyl; halogen; CN; NO₂; (C₃-C₆)cycloalkyl, halo(C₃-C₆)cycloalkyl, and hydroxy(C₃-C₆)cycloalkyl;
   wherein more than one R₁ₐ may exist, and if more than one R₁ₐ is present, then each R₁ₐ may be the same or different,
   wherein more than one R_{1b} may exist, and if more than one R_{1b} is present, then each R_{1b} may be the same or different,
   wherein R₂ is selected from hydrogen, (C₁-C₆)alkyl, halo(C₁-C₆)alkyl, hydroxy(C₁-C₆)alkyl, (C₁-C₆)alkoxy(C₁-C₆)alkyl, (C₃-C₆)cycloalkyl, halo(C₃-C₆)cycloalkyl, hydroxy(C₃-C₆)cycloalkyl, and (CH₂)₁₋₃CO₂H.

In some aspects, A₁ and A₂ are the same or different. In some aspects, R₁ₐ and R_{1b} are the same or different. In some aspects, one to six R₁ₐ groups, or any number from one to six, are present. In some aspects, one to six R_{1b} groups, or any number from one to six, are present. In some aspects, the same number of R₁ₐ and R_{1b} groups is present in the compound of formula (I).

In a second aspect, the present disclosure provides a compound of formula (II), and a method of use of these compounds. In one aspect, the present disclosure provides a method of treating, preventing, reducing the severity of, or reducing or alleviating the symptoms associated with, an eye-related disease or condition, including but not limited to cataract or presbyopia, in a subject in need thereof, the method comprising administering to the subject an effective amount of a compound having the formula (II):

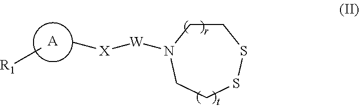

or a pharmaceutically acceptable salt thereof,
   wherein
A is a 5-10 membered heteroaryl or heteroaromatic ring comprising at least one heteroatom and at least one carbon atom, wherein A may be a monocyclic or bicyclic ring, wherein A is optionally substituted with one or more R₁ groups, wherein each R₁ is attached to one or more carbon atoms of A, and wherein when more than one R₁ is present, then each R₁ may be the same or different,
R₁ is optional, but when present, R₁ is independently selected from the group consisting of: hydrogen; (C₁-C₆)alkyl; halo(C₁-C₆)alkyl; (C₁-C₆)alkoxy; halo(C₁-C₆)alkoxy; hydroxy(C₁-C₆)alkyl; (C₁-C₆)alkoxy(C₁-C₆)alkyl; (C₂-C₅)alkenyl; (C₂-C₅)alkynyl; SO₂R₂; (CH₂)₀₋₄CO₂R₂; NH₂; NHR₂; N(R₂)(R₂) wherein each R₂ may be the same or different; (C₁-C₆)alkylamine; di(C₁-C₆)alkylamine; NHCOR₂; hydroxyl; halogen; CN; and NO₂;
   wherein R₂ is selected from: hydrogen, (C₁-C₆)alkyl, halo(C₁-C₆)alkyl, hydroxy(C₁-C₆)alkyl, (C₁-C₆)alkoxy(C₁-C₆)alkyl, (C₂-C₅)alkenyl, (C₂-C₅)alkynyl, (C₃-C₆)cycloalkyl, halo(C₃-C₆)cycloalkyl, and hydroxy(C₃-C₆)cycloalkyl;
W is a direct bond, CO, CS, or (CR_{A}R_{B})ₚ, and
X is a direct bond, O, NH, or (CR_{A}R_{B})ₚ,
   wherein each of R_{A} and R_{B} is independently selected from the group consisting of: hydrogen, halogen, and hydroxyl, wherein R_{A} and R_{B} may be the same or different,
   wherein p is any number between 0 and 4; and
each of r and t is independently any number between 1 and 3.

In some aspects, one to ten R₁ groups, or any number between one and ten, are present in the compound of formula (II).

In a third aspect, the present disclosure provides a compound of formula (III), and a method of use of these compounds. In one aspect, the present disclosure provides a method of treating, preventing, reducing the severity of, or reducing or alleviating the symptoms associated with, an eye-related disease or condition, including but not limited to cataract or presbyopia, in a subject in need thereof, the method comprising administering to the subject an effective amount of a compound having the formula (III):

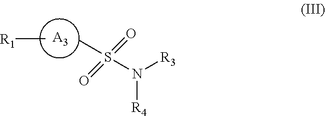

or a pharmaceutically acceptable salt thereof,
   wherein
A₃ is a 5- or 6-membered aromatic or heteroaromatic ring comprising at least one heteroatom and at least one carbon atom, wherein A₃ is optionally substituted with one or more R₁ groups, wherein each R₁ is attached to one or more carbon atoms of A₃, and wherein when more than one R₁ is present, then each R₁ may be the same or different,
R₁ is attached to one or more carbon atoms of A₃, and R₁ is independently selected from the group consisting of: hydrogen; (C₁-C₆)alkyl; halo(C₁-C₆)alkyl; (C₁-C₆)alkoxy; halo(C₁-C₆)alkoxy; hydroxy(C₁-C₆)alkyl; (C₁-C₆)alkoxy(C₁-C₆)alkyl; (C₂-C₅)alkenyl; (C₂-C₅)alkynyl; SO₂R₂; (CH₂)₀₋₄CO₂R₂; NH₂; NHR₂; N(R₂)(R₂) wherein each R₂ may be the same or different; (C₁-C₆)alkylamine; di(C₁-C₆)alkylamine; NHCOR₂; hydroxyl; halogen; CN; and NO₂;
   wherein R₂ is selected from: hydrogen, (C₁-C₆)alkyl, halo(C₁-C₆)alkyl, hydroxy(C₁-C₆)alkyl, (C₁-C₆)alkoxy(C₁-C₆)alkyl, (C₂-C₅)alkenyl, (C₂-C₅)alkynyl, (C₃-C₆)cycloalkyl, halo(C₃-C₆)cycloalkyl, and hydroxy(C₃-C₆)cycloalkyl; and
R₃ and R₄ are each independently selected from the group consisting of: hydrogen; (C₁-C₁₀)alkyl; halo(C₁-C₆)alkyl; hydroxy(C₁-C₆)alkyl; (C₁-C₆)alkoxy(C₁-C₆)alkyl; (C₂-C₅)alkenyl; (C₂-C₅)alkynyl; (C₃-C₆)cycloalkyl; halo(C₃-C₆)cycloalkyl; hydroxy(C₃-C₆)cycloalkyl; (CHR₅)ₙCO₂R₅;
or R₃ and R₄ together, with the nitrogen to which they are attached to, join together to form a 5-10 membered aromatic or heterocyclic ring or cyclic ring linked to the nitrogen with (CH₂)₁₋₄, optionally substituted at one or more ring atoms with (C₁-C₆)alkyl, halo(C₁-C₆)alkyl, hydroxy(C₁-C₆)alkyl, (C₁-C₆)alkoxy(C₁-C₆)alkyl, hydroxyl, CN, halogen, NH₂; (C₁-C₆)alkylamine; di(C₁-C₆)alkylamine; and NO₂;
   wherein R₅ is independently selected from the group consisting of: hydrogen and a (C₁-C₆)alkyl and
   wherein n is any number from 0 and 4.

In some aspects, one to six R₁ groups, or any number between one and six, are present.

In a fourth aspect, the present disclosure provides a compound of formula (IV), and a method of use of these compounds. In one aspect, the present disclosure provides a method of treating, preventing, reducing the severity of, or reducing or alleviating the symptoms associated with, an eye-related disease or condition, including but not limited to, cataract or presbyopia in a subject in need thereof, the method comprising administering to the subject an effective amount of a compound having the formula (IV):

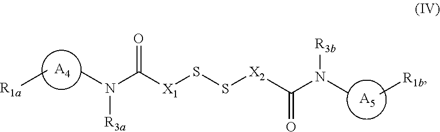

or a pharmaceutically acceptable salt thereof,
   wherein
A₄ and A₅ are each independently a 6-14 membered aromatic or heteroaromatic ring comprising at least one heteroatom and at least one carbon atom, wherein each of A₄ and A₅ are independently optionally substituted with R₁ₐ and R_{1b}, respectively,
   wherein R₁ₐ and R_{1b} are each independently optionally attached to one or more carbon atoms of the A₄ and A₅, respectively, and R₁ₐ and R_{1b} are each independently selected from the group consisting of: hydrogen; (C₁-C₆)alkyl; halo(C₁-C₆)alkyl; (C₁-C₆)alkoxy; halo(C₁-C₆)alkoxy; hydroxy(C₁-C₆)alkyl; (C₁-C₆)alkoxy(C₁-C₆)alkyl; (C₂-C₅)alkenyl; (C₂-C₅)alkynyl; CO₂R₂; NH₂; NHR₂; N(R₂)(R₂) wherein each R₂ may be the same or different (C₁-C₆)alkylamine; di(C₁-C₆)alkylamine; NHCOR₂; hydroxyl; halogen; CN; and NO₂;
   wherein R₂ is selected from the group consisting of: hydrogen, (C₁-C₆)alkyl, halo(C₁-C₆)alkyl, hydroxy(C₁-C₆)alkyl, (C₁-C₆)alkoxy(C₁-C₆)alkyl, (C₃-C₆)cycloalkyl, halo(C₃-C₆)cycloalkyl, and hydroxy(C₃-C₆)cycloalkyl;
each of R₃ₐ and R_{3b} is independently hydrogen or (C₁-C₃)alkyl; and
each of X₁ and X₂ is independently CHR₄, wherein R₄ is hydrogen or (C₁-C₃)alkyl, wherein X₁ and X₂ are the same or different.

In a fifth aspect, the present disclosure provides a compound of formula (V), and a method of use of these compounds. In one aspect, the present disclosure provides a method of treating, preventing, reducing the severity of, or reducing or alleviating the symptoms associated with, an eye-related disease or condition, including but not limited to cataract or presbyopia in a subject in need thereof, the method comprising administering to the subject an effective amount of a compound having the formula (V).

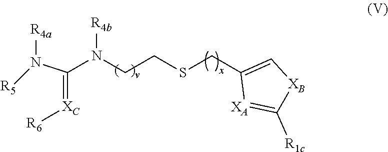

or a pharmaceutically acceptable salt thereof,
wherein:
X_{A} is N or O;
X_{B} is S or CH;
X_{C} is N or CH;
R_{1c} is selected from the group consisting of: (CH₂)₁₋₃NH₂, (CH₂)₁₋₃NH(R₂), (CH₂)₁₋₃NR₂R₃, (CH₂)₁₋₃(C₁-C₆)alkylamine, and (CH₂)₁₋₃di(C₁-C₆)alkylamine, and N═CR₂R₃, wherein R₂ and R₃ are independently hydrogen or (C₁-C₆)alkyl and may be the same or different;
R₄ₐ and R_{4b} are each independently hydrogen or (C₁-C₆)alkyl, and R₄ₐ and R_{4b} may be the same or different,
R₅ is (C₁-C₆)alkyl, (C₂-C₅)alkenyl, (C₂-C₅)alkynyl, or CH₂CN;
R₆ is NH₂, NHR₂, N(R₂)(R₂) wherein each R₂ may be the same or different, NO₂ or (C₁-C₆)alkyl, wherein R₂ is selected from the group consisting of: hydrogen, (C₁-C₆)alkyl, halo(C₁-C₆)alkyl, hydroxy(C₁-C₆)alkyl, (C₁-C₆)alkoxy(C₁-C₆)alkyl, (C₃-C₆)cycloalkyl, halo(C₃-C₆)cycloalkyl, and hydroxy(C₃-C₆)cycloalkyl;
v is any number from 0 to 3; and
x is any number from 0 to 3.

### Compounds and Definitions

The terms “halo” and “halogen” as used herein refer to an atom selected from fluorine (fluoro, —F), chlorine (chloro, —Cl), bromine (bromo, —Br), and iodine (iodo, —I).

The term “alkyl”, used alone or as a part of a larger moiety such as e.g., “haloalkyl”, means a saturated monovalent straight or branched hydrocarbon radical having, unless otherwise specified, 1-10 carbon atoms and includes, for example, methyl, ethyl, n-propyl, isopropyl, n-butyl, sec-butyl, isobutyl, tert-butyl, n-pentyl, n-hexyl, n-heptyl, n-octyl, n-nonyl, n-decyl and the like. “Monovalent” means attached to the rest of the molecule at one point.

As used herein, either alone or in combination, the term “alkenyl” refers to a functional group comprising a straight-chain or branched-chain hydrocarbon containing, unless otherwise specified, from 2 to 20 carbon atoms and having one or more carbon-carbon double bonds and not having any cyclic structure. An alkenyl group may be optionally substituted as defined herein. Examples of alkenyl groups include, without limitation, ethenyl, propenyl, 2-methylpropenyl, butenyl, 1,4-butadienyl, pentenyl, hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, and the like. The point of attachment can be on the double bond carbon or on any single bond carbon.

As used herein, either alone or in combination, the term “alkynyl” refers to a functional group comprising a straight-chain or branched-chain hydrocarbon containing, unless otherwise specified, from 2 to 20 carbon atoms and having one or more carbon-carbon triple bonds and not having any cyclic structure. An alkynyl group may be optionally substituted as defined herein. Examples of alkynyl groups include, without limitation, ethynyl, propynyl, hydroxypropynyl, butynyl, butyn-1-yl, butyn-2-yl, 3-methylbutyn-1-yl, pentynyl, pentyn-1-yl, hexynyl, hexyn-2-yl, heptynyl, octynyl, nonynyl, decynyl, undecynyl, dodecynyl, tridecynyl, tetradecynyl, pentadecynyl, hexadecynyl, heptadecynyl, octadecynyl, nonadecynyl, eicosynyl, and the like. The point of attachment can be on the triple bond carbon or on any single bond carbon.

As used herein, either alone or in combination, the term “alkoxy” refers to —O-alkyl, —O— alkenyl, or —O-alknyl, wherein alkyl, alkenyl, and alkynyl are as defined above.

As used herein, either alone or in combination, the term “alkoxyalkyl” means an alkyl as defined above substituted with an alkoxy group as defined above (in some embodiments one or two alkoxy groups). C₂₋₆ alkoxyalkyl means the total number of carbon atoms. Examples include but not limited to 2-methoxyethyl, 1-, 2-, or 3-methoxypropyl, 2-ethoxyethyl, and the like.

The terms “cycloalkyl” used alone or as part of a larger moiety, refers to a saturated cyclic aliphatic monocyclic or bicyclic ring system, as described herein, having from, unless otherwise specified, 3 to 10 carbon ring atoms. Monocyclic cycloalkyl groups include, without limitation, cyclopropyl, cyclobutyl, cyclopentyl, cyclopentenyl, cyclohexyl, cyclohexenyl, cycloheptyl, cycloheptenyl, and cyclooctyl. Bicyclic cycloalkyl groups include e.g., cycloalkyl group fused to another cycloalkyl group, such as decalin or a cycloalkyl group fused to an aryl group (e.g., phenyl) or heteroaryl group, such as tetrahydronaphthalenyl, indanyl, 5,6,7,8-tetrahydroquinoline, and 5,6,7,8-tetrahydroisoquinoline. It will be understood that the point of attachment for bicyclic cycloalkyl groups can be either on the cycloalkyl portion or on the aryl group (e.g., phenyl) or heteroaryl group that results in a stable structure. It will be further understood that when specified, optional substituents on a cycloalkyl may be present on any substitutable position and, include, e.g., the position at which the cycloalkyl is attached.

The term “heterocyclyl” means a 4-, 5-, 6- and 7-membered saturated or partially unsaturated heterocyclic ring containing 1 to 4 heteroatoms independently selected from N, O, and S. The terms “heterocycle”, “heterocyclyl”, “heterocyclyl ring”, “heterocyclic group”, “heterocyclic moiety”, and “heterocyclic radical”, may be used interchangeably. A heterocyclyl ring can be attached to its pendant group at any heteroatom or carbon atom that results in a stable structure. Examples of such saturated or partially unsaturated heterocyclic radicals include, without limitation, tetrahydrofuranyl, tetrahydrothienyl, terahydropyranyl, pyrrolidinyl, pyrrolidonyl, piperidinyl, oxetanyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, and tetrahydropyrimidinyl. A heterocyclyl group may be mono- or bicyclic. Unless otherwise specified, bicyclic heterocyclyl groups include, e.g., unsaturated or saturated heterocyclic radicals fused to another unsaturated heterocyclic radical or aromatic or heteroaryl ring, such as for example, chromanyl, 2,3-dihydrobenzo[b][1,4]dioxinyl, tetrahydronaphthyridinyl, indolinonyl, dihydropyrrolotriazolyl, imidazopynmidinyl, quinolinonyl, dioxaspirodecanyl. It will be understood that the point of attachment for bicyclic heterocyclyl groups can be on the heterocyclyl group or aromatic ring that results in a stable structure. It will also be understood that when specified, optional substituents on a heterocyclyl group may be present on any substitutable position and, include, e.g., the position at which the heterocyclyl is attached.

As used herein, either alone or in combination, the term “aryl” refers to monocyclic, bicyclic (fused), and tricyclic (fused or spiro) hydrocarbon ring system having a total of five to fourteen ring atoms. When aryl is monocyclic, the monocyclic is aromatic and contains no heteroatom. When aryl is bicyclic or tricyclic, at least one of the ring in the bicyclic or tricyclic is aromatic and contains no heteroatom, and when the other ring(s) is aromatic, the other ring(s) does not contain a heteroatom, but when the other ring(s) is not aromatic, the other ring(s) may or may not contain a heteroatom. The point of attachment can be on any ring atom. Examples of aryl include, without limitation, benzene, naphthalene, indane, 1,2,3,4-tetrahydronaphthalene, chromane, isochromane, 1,2,3,4-tetrahydroquinoline, thiochromane 1,1-dioxide, 6,7,8,9-tetrahydro-5H-benzo[7]annulene, and 2,3-dihydrobenzofuran.

As used herein, either alone or in combination, the term “aralkyl” refers to a 5 to 12 membered heteroaryl or 6 to 12 membered aryl, as defined herein, substituted for a hydrogen of an C₁₋₆ alkyl.

The term “heteroaryl” used alone or as part of a larger moiety as in “heteroarylalkyl”, “heteroarylalkoxy”, or “heteroarylaminoalkyl”, refers to a 5-10-membered aromatic radical containing 1-4 heteroatoms selected from N, O, and S and includes, for example, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The term “heteroaryl” may be used interchangeably with the terms “heteroaryl ring”, “heteroaryl group”, or “heteroaromatic”. The terms “heteroaryl” and “heteroar-” as used herein, also include groups in which a heteroaromatic ring is fused to one or more aryl rings, where the radical or point of attachment is on the heteroaromatic ring. Nonlimiting examples include indolyl, indazolyl, benzimidazolyl, benzthiazolyl, quinolyl, quinazolinyl, and quinoxalinyl. A heteroaryl group may be mono- or bicyclic. It will be understood that when specified, optional substituents on a heteroaryl group may be present on any substitutable position and, include, e.g., the position at which the heteroaryl is attached.

For any structure disclosed herein, the scope of a compound also includes any tautomer which may be formed. Unless otherwise indicated, reference to a compound should be construed broadly to include pharmaceutically acceptable salts, prodrugs, tautomers, alternate solid forms, non-covalent complexes, and combinations thereof, of a chemical entity of the depicted structure or chemical name.

A prodrug is a compound which is converted to a therapeutically active compound after administration. For example, conversion may occur by [tailor this part to the structure being claimed], or some other biologically labile group. Prodrug preparation is well known in the art. For example, “Prodrugs and Drug Delivery Systems,” which is a chapter in Richard B. Silverman, Organic Chemistry of Drug Design and Drug Action, 2d Ed., Elsevier Academic Press: Amsterdam, 2004, pp. 496-557, provides further detail on the subject.

Tautomers are isomers that are in rapid equilibrium with one another. For example, tautomers may be related by transfer of a proton, hydrogen atom, or hydride ion.

Unless stereochemistry is explicitly depicted, a structure is intended to include every possible stereoisomer, both pure or in any possible mixture.

Alternate solid forms are different solid forms than those that may result from practicing the procedures described herein. For example, alternate solid forms may be polymorphs, different kinds of amorphous solid forms, glasses, and the like.

Non-covalent complexes are complexes that may form between the compound and one or more additional chemical species that do not involve a covalent bonding interaction between the compound and the additional chemical species. They may or may not have a specific ratio between the compound and the additional chemical species. Examples might include solvates, hydrates, charge transfer complexes, and the like.

When ranges of values are disclosed, and the notation “from n₁ . . . to n₂” or “n₁ . . . to n₂” is used, where n₁ and n₂ are the numbers, then unless otherwise specified, this notation is intended to include the numbers themselves and the range between them. This range may be integral or continuous between and including the end values. By way of example, the range “3 to 11 membered cycloalkyl” is intended to include cycloalkyl having three, four, five, six, seven, eight, nine, ten, or eleven ring atoms. When n is set at 0 in the context of “0 carbon atoms”, it is intended to indicate a bond or null.

As used herein the terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The compounds described herein may be present in the form of pharmaceutically acceptable salts. For use in medicines, the salts of the compounds described herein refer to non-toxic “pharmaceutically acceptable salts.” Pharmaceutically acceptable salt forms include pharmaceutically acceptable acidic/anionic or basic/cationic salts.

Pharmaceutically acceptable basic/cationic salts include, the sodium, potassium, calcium, magnesium, diethanolamine, n-methyl-D-glucamine, L-lysine, L-arginine, ammonium, ethanolamine, piperazine, and triethanolamine salts.

Pharmaceutically acceptable acidic/anionic salts include, e.g., the acetate, benzenesulfonate, benzoate, bicarbonate, bitartrate, carbonate, citrate, dihydrochloride, gluconate, glutamate, glycollylarsanilate, hexylresorcinate, hydrobromide, hydrochloride, malate, maleate, malonate, mesylate, nitrate, salicylate, stearate, succinate, sulfate, tartrate, and tosylate.

### Description of Exemplary Compounds

In some embodiments, the present disclosure provides a compound of formula (I), pharmaceutical compositions comprising a compound of formula (I), and a method of use of the compound of formula (I):

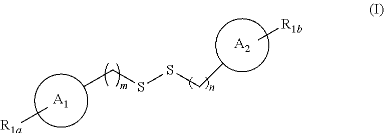

or a pharmaceutically acceptable salt thereof, wherein the variables are as described above.

In some embodiments, m and n are the same, A₁ and its R₁ₐ substituents are the same as A₂ and its R_{1b} substituents.

In some embodiments, A₁ and A₂ are the same.

In some embodiments, A₁ is a benzene ring, and at least one R₁ₐ substituent occurs at the ortho position. In some embodiments, A₁ is a benzene ring, and at least one R₁ₐ substituent occurs at the meta position. In some embodiments, A₁ is a benzene ring, and at least one R₁ₐ substituent occurs at the para position. In some embodiments, A₁ is a benzene ring, and there are two R₁ₐ substituents, wherein one R₁ₐ substituent occurs at the ortho position and one R₁ₐ substituent occurs at the para position. In some embodiments, A₁ is a benzene ring, and there are two R₁ₐ substituents, wherein one R₁ₐ substituent occurs at the ortho position and one R₁ₐ substituent occurs at the meta position. In some embodiments, A₁ is a benzene ring, and there are two R₁ₐ substituents, wherein one R₁ₐ substituent occurs at the meta position and one R₁ₐ substituent occurs at the para position. In some embodiments, A₁ is a benzene ring, and there are two R₁ₐ substituents, wherein one R₁ₐ substituent occurs at the one ortho position and one R₁ₐ substituent occurs at the other ortho position. In some embodiments, A₁ is a benzene ring, and there are two R₁ₐ substituents, wherein one R₁ₐ substituent occurs at the one meta position and one R₁ₐ substituent occurs at the other meta position.

In some embodiments, A₂ is a benzene ring, and at least one R_{1b} substituent occurs are the ortho position. In some embodiments, A₂ is a benzene ring, and at least one R_{1b} substituent occurs at the meta position. In some embodiments, A₂ is a benzene ring, and at least one R_{1b} substituent occurs at the para position. In some embodiments, A₂ is a benzene ring, and there are two R_{1b} substituents, wherein one R_{1b} substituent occurs at the ortho position and one R_{1b} substituent occurs at the para position. In some embodiments, A₂ is a benzene ring, and there are two R_{1b} substituents, wherein one R_{1b} substituent occurs at the ortho position and one R_{1b} substituent occurs at the meta position. In some embodiments, A₂ is a benzene ring, and there are two R_{1b} substituents, wherein one R_{1b} substituent occurs at the meta position and one R_{1b} substituent occurs at the para position. In some embodiments, A₂ is a benzene ring, and there are two R_{1b} substituents, wherein one R_{1b} substituent occurs at the one ortho position and one R_{1b} substituent occurs at the other ortho position. In some embodiments, A₂ is a benzene ring, and there are two R_{1b} substituents, wherein one R_{1b} substituent occurs at the one meta position and one R_{1b} substituent occurs at the other meta position.

In some embodiments, in formula (I), R₁ₐ and R_{1b} are the same or different and are each CO₂R₂, and R₂ is as defined above. In some embodiments, in formula (I), R₁ₐ and R_{1b} are the same or different and are each CO₂R₂, and R₂ is hydrogen or a (C₁-C₃)alkyl.

In some embodiments, in formula (I), R₁ₐ and R_{1b} are each CO₂R₂, R₁ₐ and R_{1b} are the same or different, and R₂ is (C₁-C₆)alkyl.

In some embodiments, in formula (I), R₁ₐ and R_{1b} are each (C₁-C₆)alkoxy, and R₁ₐ and R_{1b} are the same or different.

In some embodiments, in formula (I), R₁ₐ and R_{1b} are each NHCOR₂, and R₁ₐ and R_{1b} are the same or different, and R₂ is as defined above.

In some embodiments, in formula (I), R₁ₐ and R_{1b} are each NHCOR₂, R₁ₐ and R_{1b} are the same or different, and R₂ is (C₁-C₆)alkyl.

In some embodiments, in formula (I), R₁ₐ and R_{1b} are each SO₂R₂, R₁ₐ and R_{1b} are the same or different, and R₂ is as defined above.

In some embodiments, in formula (I), R₁ₐ and R_{1b} are each SO₂R₂, and R₁ₐ and R_{1b} are the same or different, and R₂ is halo(C₁-C₆)alkyl.

In some embodiments, in formula (I), A₁ with its R₁ₐ substitutions is identical to A₂ with its R_{1b} substitutions.

In some embodiments, the compound of formula (I) is one of

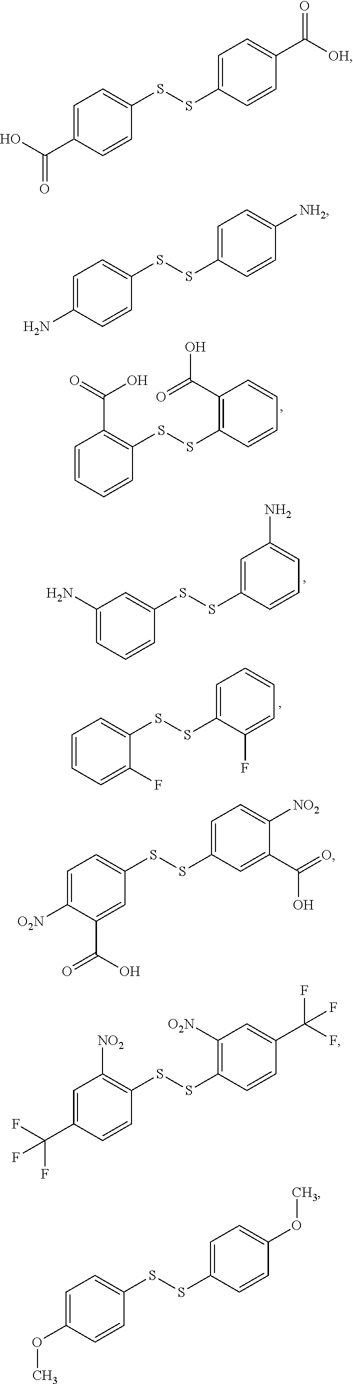

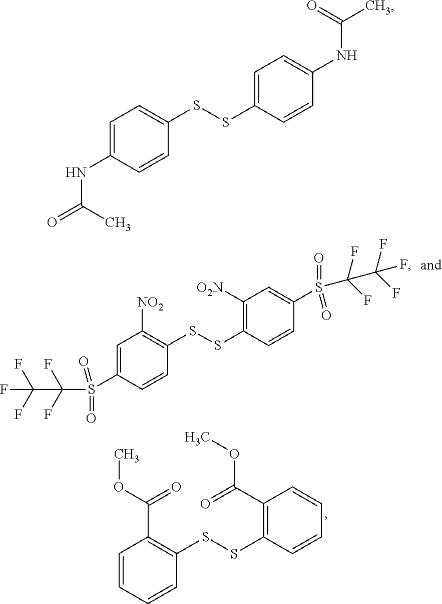

or a pharmaceutically acceptable salt thereof.

In some embodiments, the present disclosure provides a compound of formula (II), pharmaceutical compositions comprising a compound of formula (II), and a method of use of the compound of formula (II):

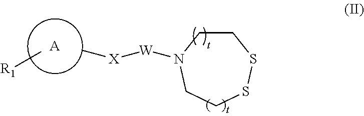

or a pharmaceutically acceptable salt thereof, wherein the variables are as described above.

In some embodiments, A is a benzene ring, and at least one R₁ substituent occurs at the ortho position. In some embodiments, A is a benzene ring, and at least one R₁ substituent occurs at the meta position. In some embodiments, A is a benzene ring, and at least one R₁ substituent occurs at the para position. In some embodiments, A is a benzene ring, and there are two R₁ substituents, wherein one R₁ substituent occurs at the ortho position and one R₁ substituent occurs at the para position. In some embodiments, A is a benzene ring, and there are two R₁ substituents, wherein one R₁ substituent occurs at the ortho position and one R₁ substituent occurs at the meta position. In some embodiments, A is a benzene ring, and there are two R₁ substituents, wherein one R₁ substituent occurs at the meta position and one R₁ substituent occurs at the para position. In some embodiments, A is a benzene ring, and there are two R₁ substituents, wherein one R₁ substituent occurs at the one ortho position and one R₁ substituent occurs at the other ortho position. In some embodiments, A is a benzene ring, and there are two R₁ substituents, wherein one R₁ substituent occurs at the one meta position and one R₁ substituent occurs at the other meta position.

In some embodiments, in formula (II), X is NH.

In some embodiments, in formula (II), W is CO.

In some embodiments, in formula (II), X and W are the same or different. In some embodiments, in formula (II), at least one of X and W represent a direct bond. In some embodiments, both X and W represent a direct bond.

In some embodiments, in formula (II), A is pyridine, pyrimidine, or pyrazine.

In some embodiments, in formula (II), A is a 5-10 membered heteroaryl or heteroaromatic ring. In some embodiments, in formula (II), A is a 5 or 6-membered heteroaromatic ring comprising at least one nitrogen heteroatom. In some embodiments, in formula (II), A is a monocyclic or bicyclic 5-10 membered heteroaryl or heteroaromatic ring.

In some embodiments, in formula (II), there are at least two R₁ groups, and R₁ is NO₂ at one position and NO₂ or (C₁-C₆)alkyl at another position.

In some embodiments, in formula (II), there are at least two R₁ groups, R₁ is NO₂ at one position and NO₂ or NH₂ at another position.

In some embodiments, in formula (II), A is pyridine.

In some embodiments, in formula (II), A is Isoindole-1,3-dione ring.

In some embodiments, in formula (II), r and t are the same or different. In some embodiments, r and t are each 1.

In some embodiments, the compound of formula (II) is one of,

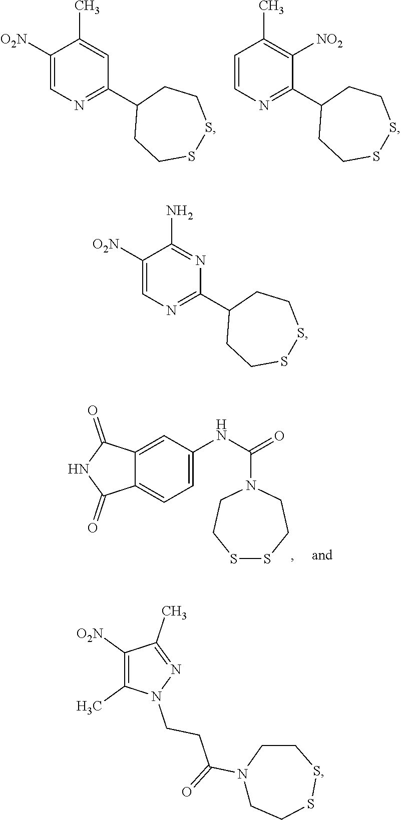

or a pharmaceutically acceptable salt thereof.

In some embodiments, the present disclosure provides a compound of formula (III), pharmaceutical compositions comprising a compound of formula (III), and a method of use of the compound of formula (III):

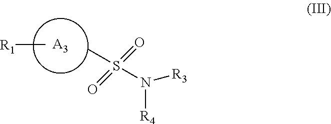

or a pharmaceutically acceptable salt thereof,
wherein the variables are as described above.

In some embodiments, A₃ is a benzene ring, and at least one R₁ substituent occurs at the ortho position. In some embodiments, A₃ is a benzene ring, and at least one R₁ substituent occurs at the meta position. In some embodiments, A₃ is a benzene ring, and at least one R₁ substituent occurs at the para position. In some embodiments, A₃ is a benzene ring, and there are two R₁ substituents, wherein one R₁ substituent occurs at the ortho position and one R₁ substituent occurs at the para position. In some embodiments, A₃ is a benzene ring, and there are two R₁ substituents, wherein one R₁ substituent occurs at the ortho position and one R₁ substituent occurs at the meta position. In some embodiments, A₃ is a benzene ring, and there are two R₁ substituents, wherein one R₁ substituent occurs at the meta position and one R₁ substituent occurs at the para position. In some embodiments, A₃ is a benzene ring, and there are two R₁ substituents, wherein one R₁ substituent occurs at the one ortho position and one R₁ substituent occurs at the other ortho position. In some embodiments, A₃ is a benzene ring, and there are two R₁ substituents, wherein one R₁ substituent occurs at the one meta position and one R₁ substituent occurs at the other meta position.

In some embodiments, in formula (III), at least one R₁ is present, and each R₁ is independently CO₂H, CO₂R₂, OH, or (CH₂)₁₋₄CO₂R₂.

In some embodiments, in formula (III), R₁ is CO₂R₂.

In some embodiments, in formula (III), at least one of R₃ and R₄ is cyclopropane.

In some embodiments, in formula (III), at least one of R₃ and R₄ is (C₁-C₆)alkyl.

In some embodiments, in formula (III), R₃ and R₄ join together to form a 5 or 6 membered heterocyclic ring, wherein the heterocyclic ring comprises a further heteroatom that is oxygen or sulfur.

In some embodiments, in formula (III), there are at least two R₁ groups, and R₁ is CO₂R₂ in one position and hydroxyl at another position.

In some embodiments, A₃ is a benzene ring.

In some embodiments, A₃ is thiophene.

In some embodiments, the compound of formula (III) is one of

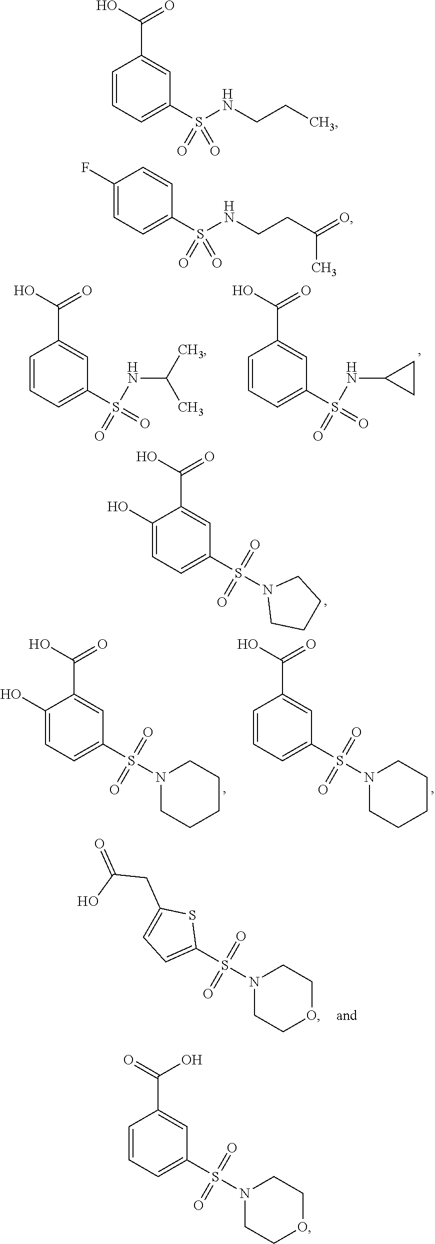

or a pharmaceutically acceptable salt thereof.

In some embodiments, the present disclosure provides a compound of formula (IV), pharmaceutical compositions comprising a compound of formula (IV), and a method of use of the compound of formula (IV):

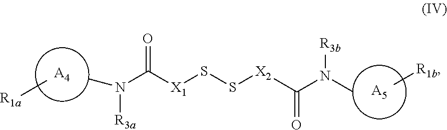

or a pharmaceutically acceptable salt thereof, wherein the variable are as described above.

In some embodiments, A₄ is a benzene ring, and at least one R₁ₐ substituent occurs at the ortho position. In some embodiments, A₄ is a benzene ring, and at least one R₁ₐ substituent occurs at the meta position. In some embodiments, A₄ is a benzene ring, and at least one R₁ₐ substituent occurs at the para position. In some embodiments, A₄ is a benzene ring, and there are two R₁ₐ substituents, wherein one R₁ₐ substituent occurs at the ortho position and one R₁ₐ substituent occurs at the para position. In some embodiments, A₄ is a benzene ring, and there are two R₁ₐ substituents, wherein one R₁ₐ substituent occurs at the ortho position and one R₁ₐ substituent occurs at the meta position. In some embodiments, A₄ is a benzene ring, and there are two R₁ₐ substituents, wherein one R₁ₐ substituent occurs at the meta position and one R₁ₐ substituent occurs at the para position. In some embodiments, A₄ is a benzene ring, and there are two R₁ₐ substituents, wherein one R₁ₐ substituent occurs at the one ortho position and one R₁ₐ substituent occurs at the other ortho position. In some embodiments, A₄ is a benzene ring, and there are two R₁ₐ substituents, wherein one R₁ₐ substituent occurs at the one meta position and one R₁ₐ substituent occurs at the other meta position.

In some embodiments, A₅ is a benzene ring, and at least one R_{1b} substituent occurs are the ortho position. In some embodiments, A₅ is a benzene ring, and at least one R_{1b} substituent occurs at the meta position. In some embodiments, A₅ is a benzene ring, and at least one R_{1b} substituent occurs at the para position. In some embodiments, A₅ is a benzene ring, and there are two R_{1b} substituents, wherein one R_{1b} substituent occurs at the ortho position and one R_{1b} substituent occurs at the para position. In some embodiments, A₅ is a benzene ring, and there are two R_{1b} substituents, wherein one R_{1b} substituent occurs at the ortho position and one R_{1b} substituent occurs at the meta position. In some embodiments, A₅ is a benzene ring, and there are two R_{1b} substituents, wherein one R_{1b} substituent occurs at the meta position and one R_{1b} substituent occurs at the para position. In some embodiments, A₅ is a benzene ring, and there are two R_{1b} substituents, wherein one R_{1b} substituent occurs at the one ortho position and one R_{1b} substituent occurs at the other ortho position. In some embodiments, A₅ is a benzene ring, and there are two R_{1b} substituents, wherein one R_{1b} substituent occurs at the one meta position and one R_{1b} substituent occurs at the other meta position.

In some embodiments, in formula (IV), A₄ and A₅ are the same or different, and are each independently phenyl or napthyl.

In some embodiments, in formula (IV), one or both of R₁ₐ and R_{1b} is NO₂.

In some embodiments, in formula (IV), there are at least two R₁ₐ groups, and R₁ₐ is a halogen at one position and (C₁-C₆)alkyl at another position. In some embodiments, in formula (IV), there are at least two R_{1b} groups, and R_{1b} is a halogen at one position and (C₁-C₆)alkyl at another position.

In some embodiments, in formula (IV), R₁ₐ is a halogen and R_{1b} is a (C₁-C₆)alkyl. In some embodiments, in formula (IV), R₁ₐ is a (C₁-C₆)alkyl and R_{1b} is a halogen.

In some embodiments, in formula (IV), X₁ and X₂ are the same.

In some embodiments, in formula (IV), one or both of R₃ₐ and R_{3b} is hydrogen.

In some embodiments, in formula (IV), R₄ in one or both of X₁ and X₂ is methyl.

In some embodiments, in formula (IV), A₄ with its R₁ₐ substitutions is identical to A₅ with its R_{1b} substitutions.

In some embodiments, in formula (IV), X₁ and X₂ are the same, and A₄ with its R₁ₐ substitutions is identical to A₅ with its R_{1b} substitutions.

In some embodiments, in formula (IV), X₁ and X₂ are the same; R₃ₐ and R_{3b} are the same; and A₄ with its R₁ₐ substitutions is identical to A₅ with its R_{1b} substitutions.

In some embodiments, the compound of formula (IV) is one of

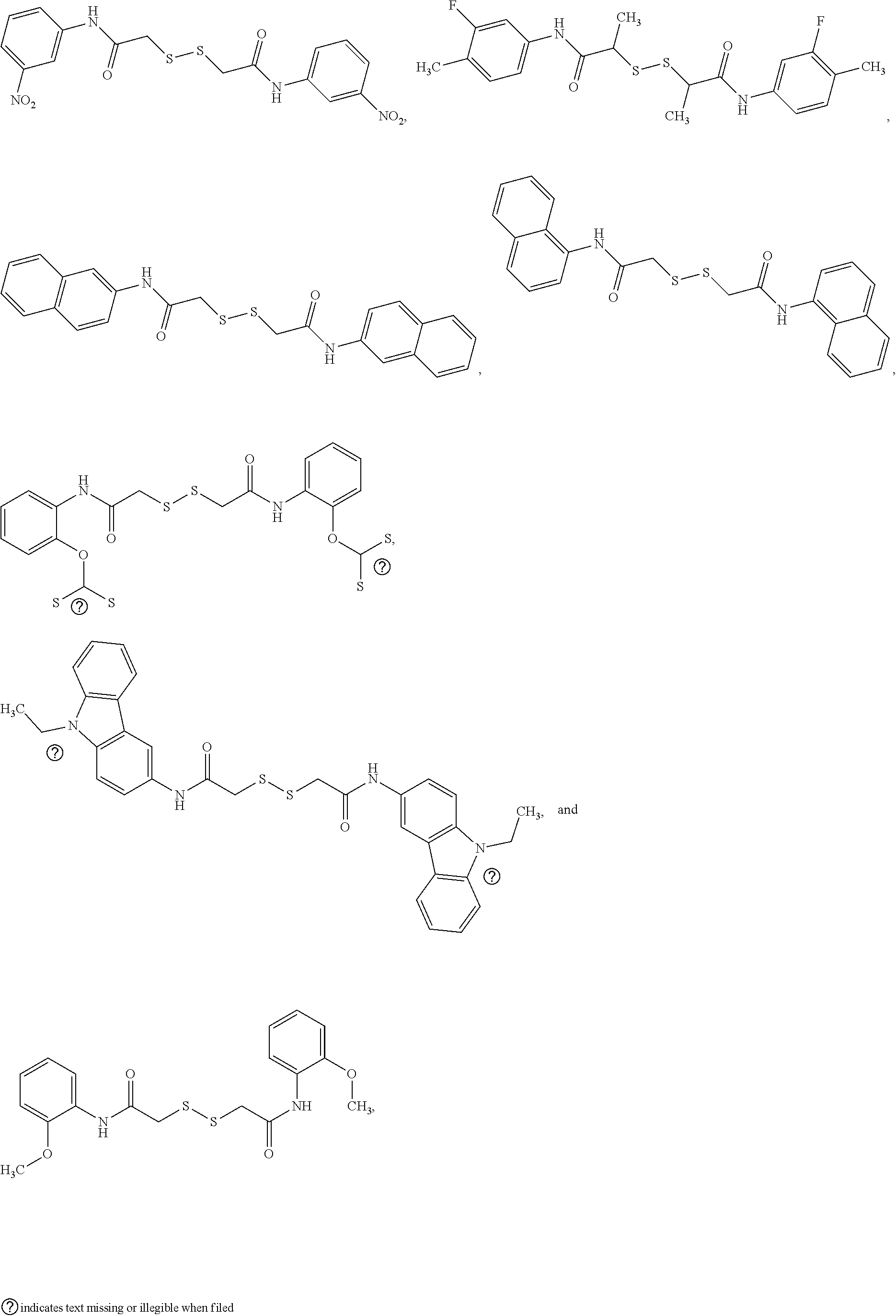

or a pharmaceutically acceptable salt thereof.

In some embodiments, the present disclosure provides a compound of formula (V), pharmaceutical compositions comprising a compound of formula (V), and a method of use of the compound of formula (V):

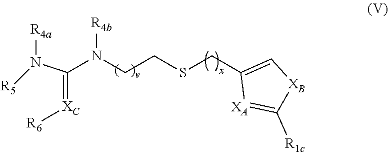

or a pharmaceutically acceptable salt thereof, wherein the variables are as described above.

In some embodiments, in formula (V), X_{A} is N and X_{B} is S.

In some embodiments, in formula (V), X_{A} is O and X_{B} is CH.

In some embodiments, in formula (V), R₅ is CN.

In some embodiments, in formula (V), R₅ is (C₁-C₆)alkyl.

In some embodiments, in formula (V), R₆ is (C₁-C₆)alkyl, or a (C₁-C₃)alkyl.

In some embodiments, in formula (V), R₆ is NO₂.

In some embodiments, in formula (V), R₄ₐ and R_{4b} are the same.

In some embodiments, in formula (V), at least one of R₄ₐ and R₅ is hydrogen.

In some embodiments, in formula (V), v is 2, and x is 1.

In some embodiments, in formula (V), X_{A} is N; X_{B} is S; v is 2, and x is 1; X_{C} is CH, R₄ₐ is a (C₁-C₃) alkyl; R₅ is hydrogen; R₆ is NO₂, NH₂, NHR₂R₃, or CH₃; and R_{1c} is CHNHR₂ or CHNR₂R₃, wherein R₂ and R₃ are the same or different and are each independently (C₁-C₃)alkyl.

In some embodiments, in formula (V), X_{A} is O; X_{B} is CH; v is 2, and x is 1; X_{C} is CH, R₄ₐ is a (C₁-C₃) alkyl; R₅ is hydrogen; R₆ is NO₂, NH₂, NHR₂R₃, or CH₃; and R_{1c} is CHNHR₂ or CHNR₂R₃, wherein R₂ and R₃ are the same or different and are each independently (C₁-C₃)alkyl.

In some embodiments, in formula (V), X_{A} is O; X_{B} is CH; v is 2, and x is 1; X_{C} is N, R₄ₐ is hydrogen; R₅ is CH₂CN; R₆ is (C₁-C₃)alkyl; and R_{1c} is N═CR₂R₃, wherein R₂ and R₃ are the same or different and are each independently (C₁-C₃)alkyl or hydrogen.

In some embodiments, the compound of formula (V) is one of

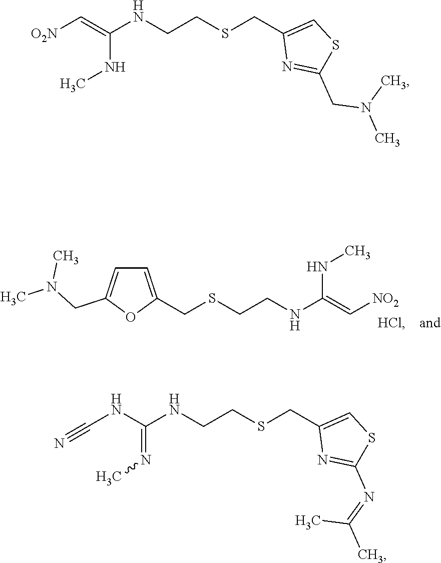

or a pharmaceutically acceptable salt thereof.

In one aspect, the compounds of the present disclosure can be produced by the following general methods.

### General Methods for Obtaining Compounds of the Present Disclosure

Reaction Schemes A, B, C and D illustrate general methods for obtaining the compounds of present disclosure.

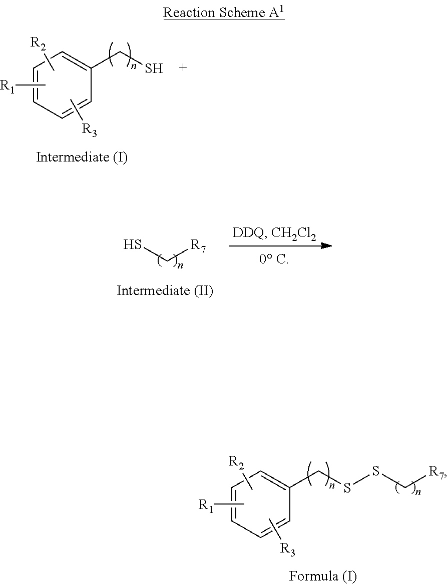

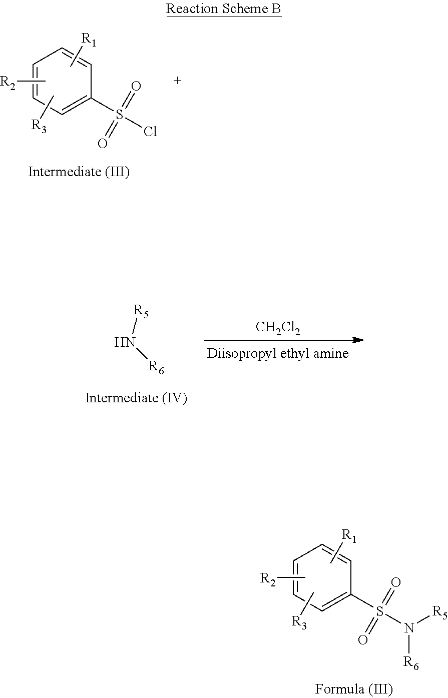

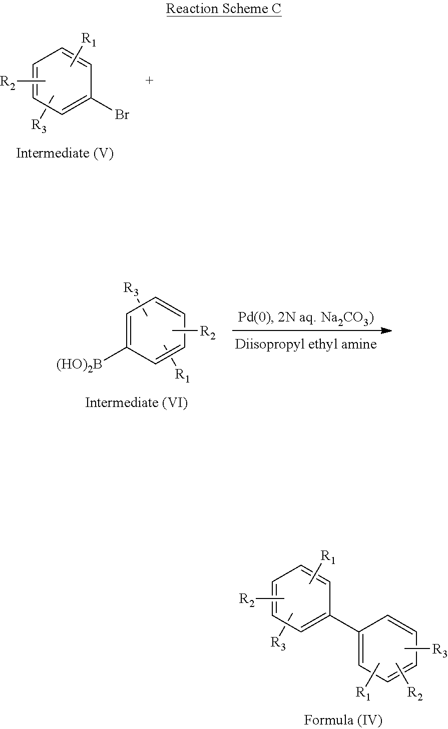

### REF

1. J. K. Vandavasi et al., Tetrahedron 67 (2011), p. 8895-8901

Further, U.S. Pat. No. 5,700,945, which is incorporated by reference in its entirety, discloses method of making compounds of Formula (V).

### Prodrug Synthesis Example

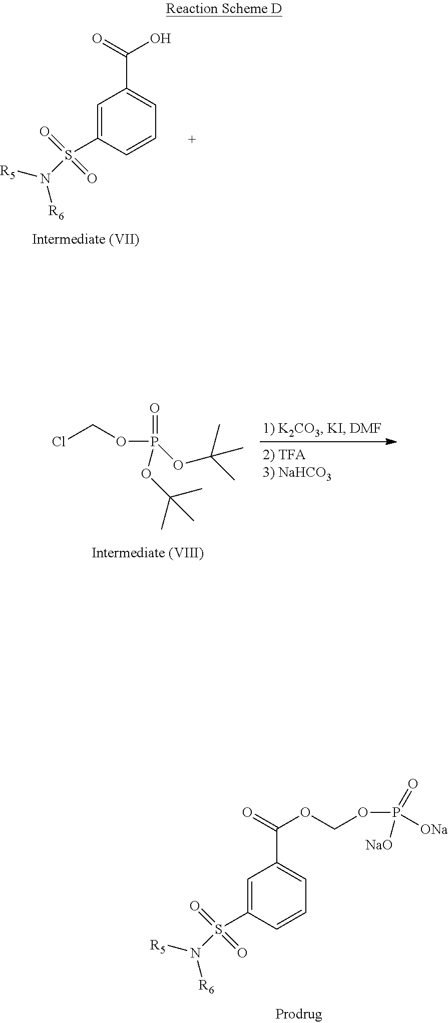

In one aspect, provided is a method of treating, preventing, reducing the occurrence of, or reducing, ameliorating, or alleviating the symptoms associated with presbyopia, cataract, transthyretin (TTR)-associated amyloidosis, or other conditions or disorders associated with the eye, comprising administering to a subject in need thereof, an effective amount of the compounds of the present disclosure, including the disclosed compounds of formula (I), (II), (III), (IV), and (V), or pharmaceutically acceptable salts thereof, or prodrugs thereof, or any of the disclosed compounds. In some aspects, provided is a pharmaceutical composition the disclosed compounds of formula (I), (II), (III), (IV), and (V), or pharmaceutically acceptable salts thereof, or prodrugs thereof, or any of the disclosed compounds, and one or more pharmaceutically excipients.

In some aspects, the compounds of the present disclosure may be administered through any route of administration, including but not limited to oral, nasal, intranasal, intramuscular, intravenous, subcutaneous, rectal, sublingual, intrathecal, transdermal, intraocularly, inhalation or other topical. In some aspects, the compounds are administrated intraocularly or topically to the eye. In some aspects, the pharmaceutically composition is an ophthalmic solution or suspension comprising the compound and one or more pharmaceutically acceptable excipients suitable for administration to the eye.

### EXAMPLES

### Example 1: Measurement of Biochemical Potency of Compounds

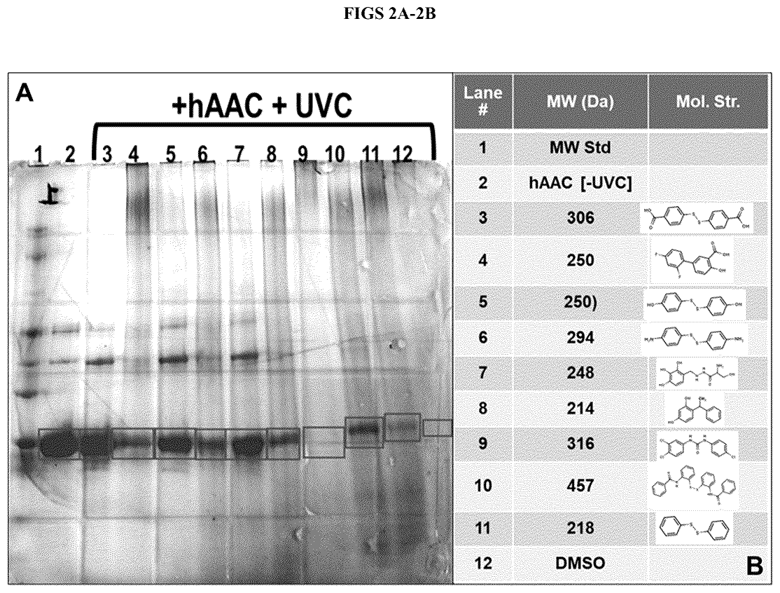

The potency of compounds to provide protection from Ca⁺² and UVC induced aggregation of alpha-A crystalline (ACC) was measured by monitoring the effect of Ca⁺² and UVC on recombinant human hAAC. hAAC forms HMW aggregates when exposed to UVC radiation in the presence of 10 mM Ca⁺². The aggregates lead to increased absorbance. Stock compound solutions (made in DMSO) were added to recombinant hAAC (100 ug/ml) at desired concentrations. The hAAC, with the added compound, was incubated at room temperature for 1 hour, followed by addition of 10 μl of 200 mM CaCl₂) and further incubation for 5 minutes. Then, absorbance was recorded at 400 nM (T=0 Minute) followed by exposure to UVC (254 nm, 480 mJ/cm² per minute). Absorbance was measured again at 15 and 30 minutes. To control for compound related effects, assays were performed with or without AAC in triplicate. Percent protection or change in fold-decrease in aggregation were computed using the measured differences in absorbance. DMSO was used as control. Results obtained are shown below in Table 1. EC₅₀ values were calculated using the SDS-PAGE, which is defined as the concentration of SMD needed to prevent 50% of hAAC from forming HMW aggregates when exposed to UVC in presence of Ca⁺² [FIGS. 2A-2B].

### Example 2: Cellular Activity of AC Based Evaluation and Optimization of SMDs

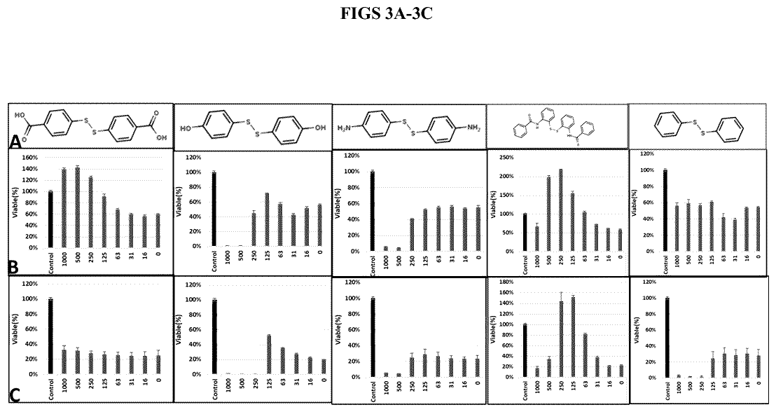

A mild heating assay using human lens epithelial (HLE) cells has been previously used as a model system for mimicking lens aging, where the time of heating is correlated with a loss in AC function and cell death. SRA 01/04 cells were pre-incubated for 2 hours with or without compounds and then exposed to UV light or heat and cell viability was assessed by Alamar blue 24-hours after exposure. The percent viability was calculated relative to untreated drug [FIGS. 3A-3C]. Correspondingly, heating of intact human lenses dramatically promotes conversion of soluble AC into HMW aggregates and induces opacity.

### Example 3: Ex Vivo Lens Culture-Based Evaluation and Optimization of SMDs

To predict whether SMDs could prevent opacification in animal models and ultimately in human patients, we assessed their ability to protect intact lenses cultured ex vivo from UV-induced opacification. As fresh human lenses are difficult to obtain, porcine lenses were used for the ex vivo studies. Porcine lenses are easy to acquire (Sierra for Medical Science, Inc., Whittier, CA) and have been used previously in ex vivo models of Presbyopia. As predicted by our biochemical assays, SMDs prevents UV-induced opacification of porcine lenses ex vivo.

TABLE 1
Compound with data

| | | EC₅₀ | | | Cell | Cell Heat |
|---|---|---|---|---|---|---|
| | | Bio- | | Ex- | UV % | % |
| ID # | Structure | chemical | Gel | vivo | protection | protection |
| 1 | 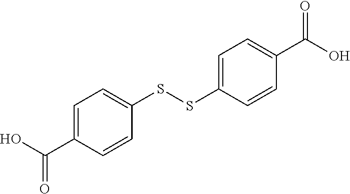 [SMILES: O=C(O)c1ccc(SSc2ccc(C(=O)O)cc2)cc1] | | ++ | | 116 | 22 |
| 2 | 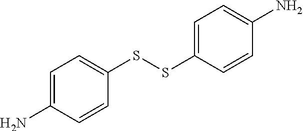 [SMILES: Nc1ccc(SSc2ccc(N)cc2)cc1] | | ++ | | | |
| 3 | 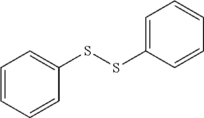 [SMILES: c1ccc(SSc2ccccc2)cc1] | | − | | 4 | n/a |
| 4 | 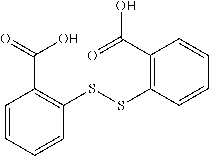 [SMILES: O=C(O)c1ccccc1SSc1ccccc1C(=O)O] | 395 uM | ++ | − | 103 | 26 |
| 5 | 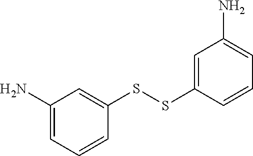 [SMILES: Nc1cccc(SSc2cccc(N)c2)c1] | | ++ | | n/a | n/a |
| 6 | 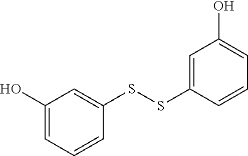 [SMILES: Oc1cccc(SSc2cccc(O)c2)c1] | | − | | n/a | n/a |
| 7 | 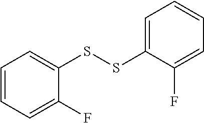 [SMILES: Fc1ccccc1SSc1ccccc1F] | | + | | n/a | n/a |
| 8 | 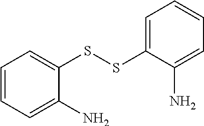 [SMILES: Nc1ccccc1SSc1ccccc1N] | | − | | n/a | n/a |
| 9 | 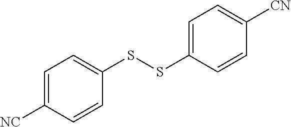 [SMILES: N#Cc1ccc(SSc2ccc(C#N)cc2)cc1] | | − | | | |
| 10 | 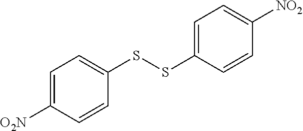 [SMILES: O=[N+]([O-])c1ccc(SSc2ccc([N+](=O)[O-])cc2)cc1] | | − | | | |
| 11 | 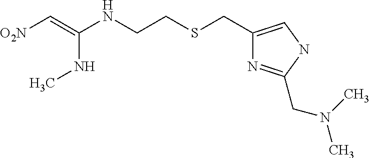 [SMILES: CN/C(=C\[N+](=O)[O-])NCCSCc1c[nH]c(CN(C)C)n1] | 495 uM | ++ | ++ | 57 | n/a |
| 12 | 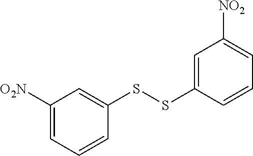 [SMILES: O=[N+]([O-])c1cccc(SSc2cccc([N+](=O)[O-])c2)c1] | | − | | | |
| 13 | 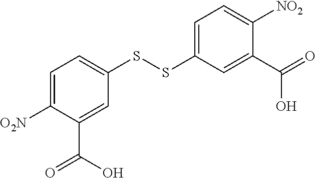 [SMILES: O=C(O)c1cc(SSc2ccc([N+](=O)[O-])c(C(=O)O)c2)ccc1[N+](=O)[O-]] | 191 uM | ++ | ++ | 52 | 2 |
| 14 | 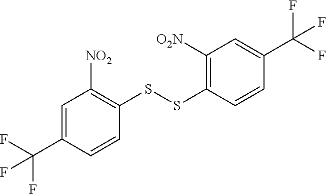 [SMILES: O=[N+]([O-])c1cc(C(F)(F)F)ccc1SSc1ccc(C(F)(F)F)cc1[N+](=O)[O-]] | | + | | n/a | n/a |
| 15 | 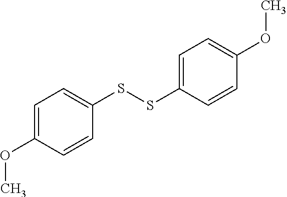 [SMILES: COc1ccc(SSc2ccc(OC)cc2)cc1] | | + | | 7 | n/a |
| 16 | 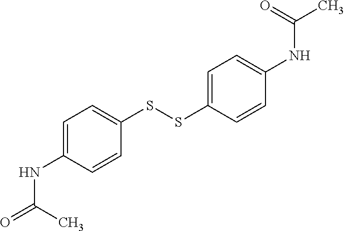 [SMILES: CC(=O)Nc1ccc(SSc2ccc(NC(C)=O)cc2)cc1] | | + | | 16 | 54 |
| 17 | 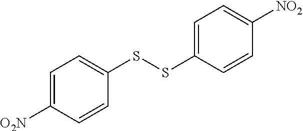 [SMILES: O=[N+]([O-])c1ccc(SSc2ccc([N+](=O)[O-])cc2)cc1] | | − | | 17 | 14 |
| 18 | 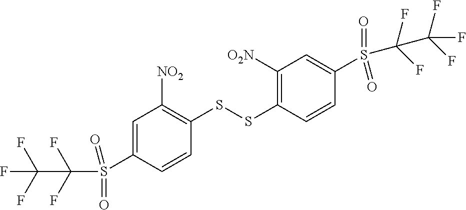 [SMILES: O=[N+]([O-])c1cc(S(=O)(=O)C(F)(F)C(F)(F)F)ccc1SSc1ccc(S(=O)(=O)C(F)(F)C(F)(F)F)cc1[N+](=O)[O-]] | | + | | 20 | n/a |
| 19 | 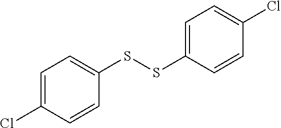 [SMILES: Clc1ccc(SSc2ccc(Cl)cc2)cc1] | | − | | | |
| 20 | 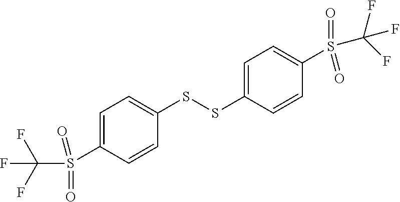 [SMILES: O=S(=O)(c1ccc(SSc2ccc(S(=O)(=O)C(F)(F)F)cc2)cc1)C(F)(F)F] | | − | | | |
| 21 | 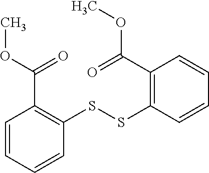 [SMILES: COC(=O)c1ccccc1SSc1ccccc1C(=O)OC] | | + | | 52 | 29 |
| 22 | 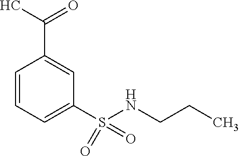 [SMILES: CCCNS(=O)(=O)c1cccc(C(C)=O)c1] | | + | | n/a | n/a |
| 23 | 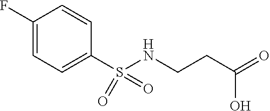 [SMILES: O=C(O)CCNS(=O)(=O)c1ccc(F)cc1] | | + | | n/a | n/a |
| 24 | 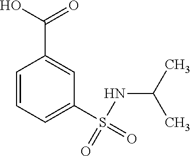 [SMILES: CC(C)NS(=O)(=O)c1cccc(C(=O)O)c1] | | + | ++ | n/a | 5 |
| 25 | 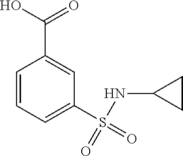 [SMILES: O=C(O)c1cccc(S(=O)(=O)NC2CC2)c1] | | + | ++ | n/a | 2 |
| 26 | 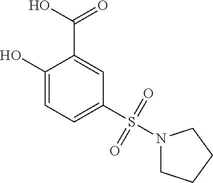 [SMILES: O=C(O)c1cc(S(=O)(=O)N2CCCC2)ccc1O] | | ++ | − | 62 | 9 |
| 27 | 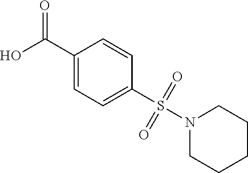 [SMILES: O=C(O)c1ccc(S(=O)(=O)N2CCCCC2)cc1] | | + | | 39 | 8 |
| 28 | 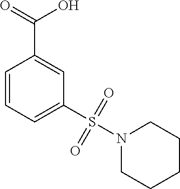 [SMILES: O=C(O)c1cccc(S(=O)(=O)N2CCCCC2)c1] | | + | | 4 | |
| 29 | 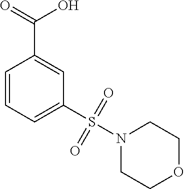 [SMILES: O=C(O)c1cccc(S(=O)(=O)N2CCOCC2)c1] | | + | | 4 | |
| 30 | 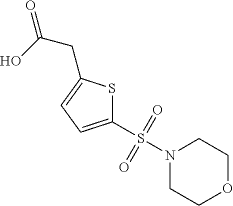 [SMILES: O=C(O)Cc1ccc(S(=O)(=O)N2CCOCC2)s1] | | ++ | | 56 | 9 |
| 31 | 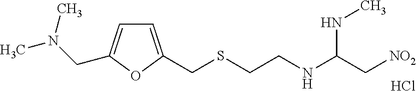 [SMILES: CNC(C[N+](=O)[O-])NCCSCc1ccc(CN(C)C)o1 ; Cl] | | + | | 49 | |
| 32 | 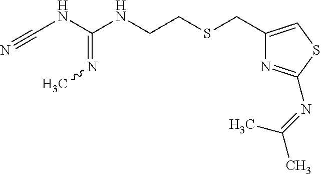 | 410 uM | ++ | | 24 | 10 |
| 33 | 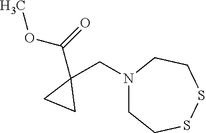 | | − | | n/a | 9 |
| 34 | 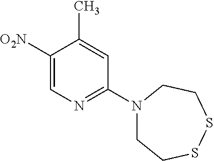 [SMILES: Cc1cc(N2CCSSCC2)ncc1[N+](=O)[O-]] | | ++ | | n/a | 9 |
| 35 | 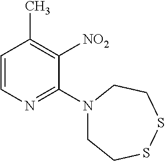 [SMILES: Cc1ccnc(N2CCSSCC2)c1[N+](=O)[O-]] | | ++ | | n/a | n/a |
| 36 | 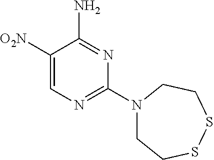 [SMILES: Nc1nc(N2CCSSCC2)ncc1[N+](=O)[O-]] | | + | | 8 | |
| 37 | 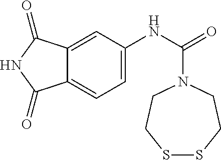 [SMILES: O=C1NC(=O)c2cc(NC(=O)N3CCSSCC3)ccc21] | 370 uM | ++ | | 76 | 3 |
| 38 | 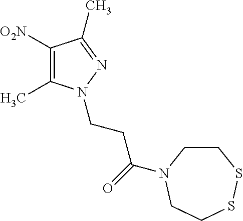 [SMILES: Cc1nn(CCC(=O)N2CCSSCC2)c(C)c1[N+](=O)[O-]] | | + | | 22 | |
| 39 | 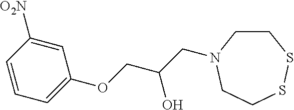 [SMILES: O=[N+]([O-])c1cccc(OCC(O)CN2CCSSCC2)c1] | | − | | | |
| 40 | 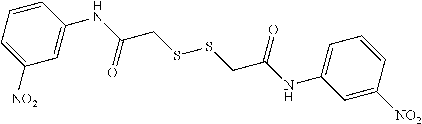 [SMILES: O=C(CSSCC(=O)Nc1cccc([N+](=O)[O-])c1)Nc1cccc([N+](=O)[O-])c1] | 365 uM | + | | 17 | n/a |
| 41 | 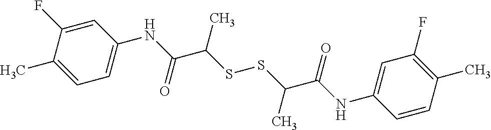 | | + | | n/a | n/a |
| 42 | 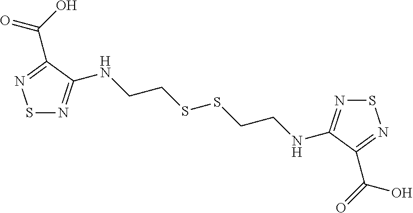 [SMILES: O=C(O)c1nsnc1NCCSSCCNc1nsnc1C(=O)O] | | + | | 24 | 13 |
| 43 | 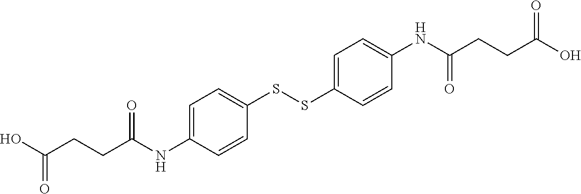 [SMILES: O=C(O)CCC(=O)Nc1ccc(SSc2ccc(NC(=O)CCC(=O)O)cc2)cc1] | | ++ | | 85 | 15 |
| 44 | 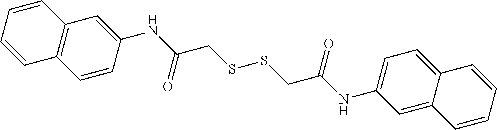 [SMILES: O=C(CSSCC(=O)Nc1ccc2ccccc2c1)Nc1ccc2ccccc2c1] | | ++ | | n/a | 3 |
| 45 | 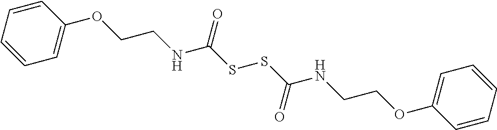 [SMILES: O=C(NCCOc1ccccc1)SSC(=O)NCCOc1ccccc1] | | + | | 15 | n/a |
| 46 | 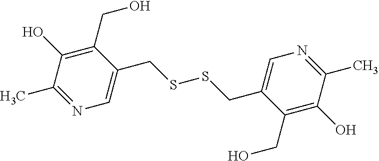 | | + | | 48 | 2 |
| 47 | 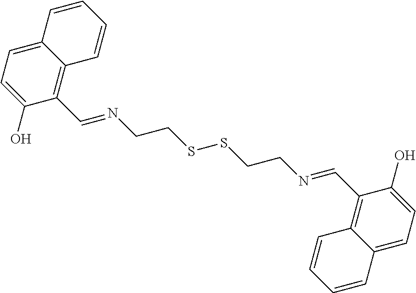 [SMILES: Oc1ccc2ccccc2c1/C=N/CCSSCC/N=C/c1c(O)ccc2ccccc12] | | − | | | |
| 48 | 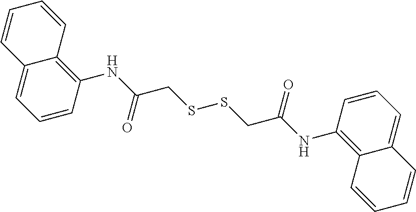 [SMILES: O=C(CSSCC(=O)Nc1cccc2ccccc12)Nc1cccc2ccccc12] | | + | | 1 | |
| 49 | 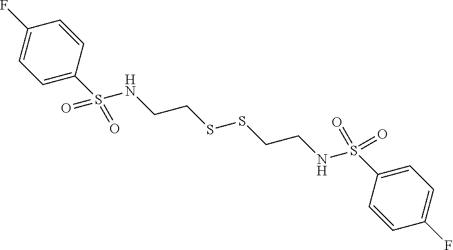 [SMILES: O=S(=O)(NCCSSCCNS(=O)(=O)c1ccc(F)cc1)c1ccc(F)cc1] | | − | | | |
| 50 | 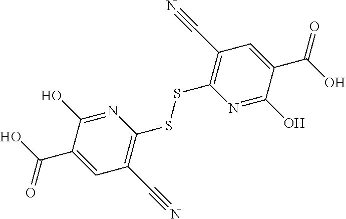 [SMILES: N#Cc1cc(C(=O)O)c(O)nc1SSc1nc(O)c(C(=O)O)cc1C#N] | | + | | 66 | |
| 51 | 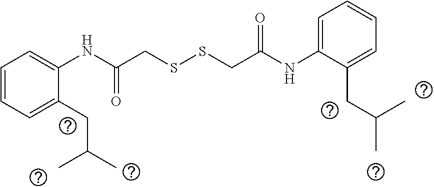 [SMILES: CC(C)Cc1ccccc1NC(=O)CSSCC(=O)Nc1ccccc1CC(C)C] | | ++ | | n/a | |
| 52 | 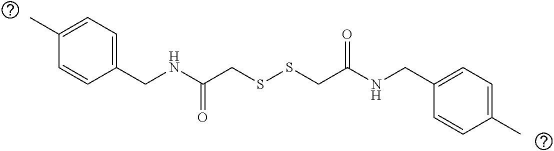 | | − | | | |
| 53 | 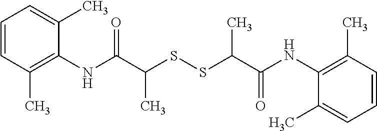 [SMILES: Cc1cccc(C)c1NC(=O)C(C)SSC(C)C(=O)Nc1c(C)cccc1C] | | − | | | |
| 54 | 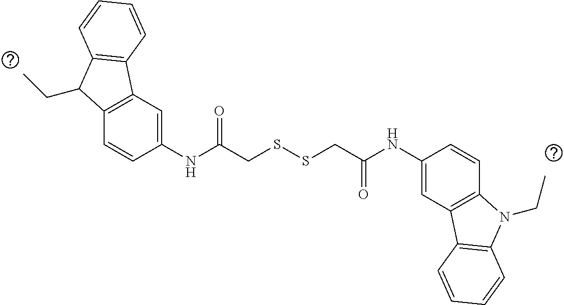 [SMILES: CCC1c2ccccc2-c2cc(NC(=O)CSSCC(=O)Nc3ccc4c(c3)c3ccccc3n4CC)ccc21] | | ++ | | n/a | |
| 55 | 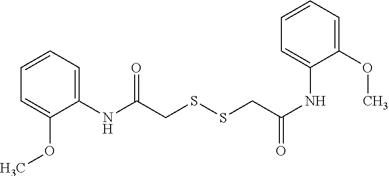 [SMILES: COc1ccccc1NC(=O)CSSCC(=O)Nc1ccccc1OC] | | ++ | | 22 | |

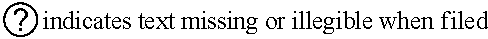

Note:
″-″ = 20% or less unaggregated AAC
″+″ score = >20% to 50% unaggregated AAC
″++″ score = >50% unaggregated AAC
Cell data: % activity at 500 uM; n/a = not active

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the described compounds, products and methods, and without departing from the spirit and scope thereof, can make various changes and modifications thereof to adapt it to various usages and conditions. Thus, other embodiments are also within the scope of the following claims.

1. A compound of formula (I): 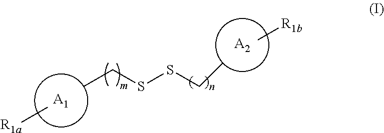
or a pharmaceutically acceptable salt thereof,
wherein
A₁ and A₂ are, each independently, a six-membered aromatic or heteroaromatic ring comprising at least one heteroatom and at least one carbon atom;
each of m and n is, independently, any number from 0 to 4;
R₁ₐ is attached to one or more carbon atoms of A₁; and R_{1b} is attached to one or more carbon atoms of A₂,
R₁ₐ and R_{1b} are, each independently, selected from the group consisting of: hydrogen; (C₁-C₆)alkyl; halo(C₁-C₆)alkyl; (C₁-C₆)alkoxy; halo(C₁-C₆)alkoxy; hydroxy(C₁-C₆)alkyl; (C₁-C₆)alkoxy(C₁-C₆)alkyl; (C₂-C₅)alkenyl; (C₂-C₅)alkynyl; SO₂R₂; CO₂R₂; NH₂; (R₂)(R₂) wherein each R₂ may be the same or different, (C₁-C₆)alkylamine; (C₁-C₆)dialkylamine; NHCOR₂; hydroxyl; halogen; CN; NO₂; (C₃-C₆)cycloalkyl, halo(C₃-C₆)cycloalkyl, and hydroxy(C₃-C₆)cycloalkyl;
wherein more than one R₁ₐ may exist, and if more than one R₁ₐ is present, then each R₁ₐ may be the same or different,
wherein more than one R_{1b} may exist, and if more than one R_{1b} is present, then each R_{1b} may be the same or different, and
wherein R₂ is selected from hydrogen, (C₁-C₆)alkyl, halo(C₁-C₆)alkyl, hydroxy(C₁-C₆)alkyl, (C₁-C₆)alkoxy(C₁-C₆)alkyl, (C₃-C₆)cycloalkyl, halo(C₃-C₆)cycloalkyl, hydroxy(C₃-C₆)cycloalkyl, and (CH₂)₁₋₃CO₂H.

2. The compound of claim 1, wherein R₁ₐ and R_{1b} are the same or different and are each independently CO₂R₂, NHCOR₂, or SO₂R₂.

3. The compound of claim 1, wherein R₂ is (C₁-C₆)alkyl or halo(C₁-C₆)alkyl.

4. The compound of claim 1, wherein R₁ₐ and R_{1b} are each (C₁-C₆)alkoxy, and R₁ₐ and R_{1b} are the same or different.

5. The compound of claim 1, wherein A1 with its R₁ₐ substitutions is identical to A₂ with its R_{1b} substitutions.

6. The compound of claim 1, wherein A1 is benzene.

7. A compound selected from the group consisting of: 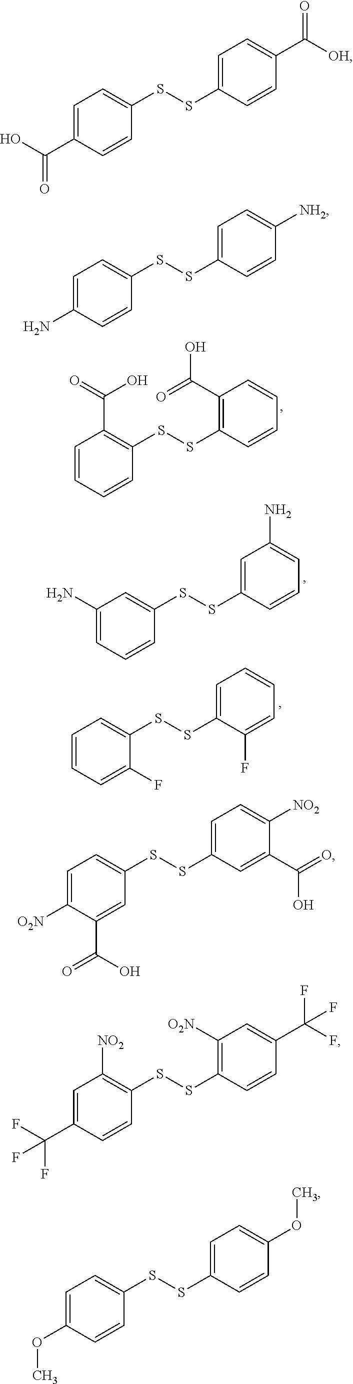 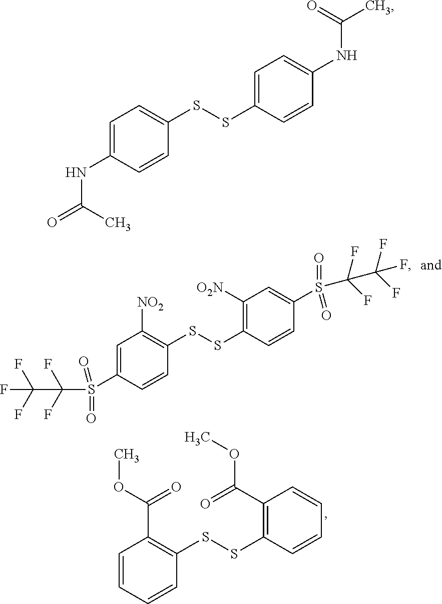
or a pharmaceutically acceptable salt thereof.

8. A compound of formula (II) 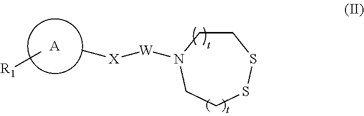
or a pharmaceutically acceptable salt thereof,
wherein
A is a 5-10 membered heteroaryl or heteroaromatic ring comprising at least one heteroatom and at least one carbon atom, wherein A may be a monocyclic or bicyclic ring, wherein A is optionally substituted with one or more R₁ groups, wherein each R₁ is attached to one or more carbon atoms of A, and wherein when more than one R₁ is present, then each R₁ may be the same or different,
R₁ is optional, but when present, R₁ is independently selected from the group consisting of: hydrogen; (C₁-C₆)alkyl; halo(C₁-C₆)alkyl; (C₁-C₆)alkoxy; halo(C₁-C₆)alkoxy; hydroxy(C₁-C₆)alkyl; (C₁-C₆)alkoxy(C₁-C₆)alkyl; (C₂-C₅)alkenyl; (C₂-C₅)alkynyl; SO₂R₂; (CH₂)₀₋₄CO₂R₂; NH₂; NHR₂; N(R₂)(R₂) wherein each R₂ may be the same or different; (C₁-C₆)alkylamine; di(C₁-C₆)alkylamine; NHCOR₂; hydroxyl; halogen; CN; and NO₂;
wherein R₂ is selected from: hydrogen, (C₁-C₆)alkyl, halo(C₁-C₆)alkyl, hydroxy(C₁-C₆)alkyl, (C₁-C₆)alkoxy(C₁-C₆)alkyl, (C₂-C₅)alkenyl, (C₁-C₅)alkynyl, (C₃-C₆)cycloalkyl, halo(C₃-C₆)cycloalkyl, and hydroxy(C₃-C₆)cycloalkyl;
W is a direct bond, CO, CS, or (CR_{A}R_{B})ₚ, and
X is a direct bond, 0, NH, or (CR_{A}R_{B})ₚ,
wherein each of R_{A} and R_{B} is independently selected from the group consisting of: hydrogen, halogen, and hydroxyl, wherein R_{A} and R_{B} may be the same or different,
wherein p is any number between 0 and 4; and
each of r and t is independently any number between 1 and 3.

9. The compound of claim 8, wherein X is NH or a direct bond and W is CO or a direct bond.

10. The compound of claim 8, wherein A is pyridine, pyrimidine, pyrazine, or isoindole-1,3-dione.

11. The compound of claim 8, wherein A is a 5-10 membered heteroaryl or heteroaromatic monocyclic or bicyclic ring.

12. The compound of claim 8, wherein there are at least two R₁ groups, and R₁ is NO₂ at one position and NO₂, NH₂, or (C₁-C₆)alkyl at another position.

13. A compound selected from the group consisting of: 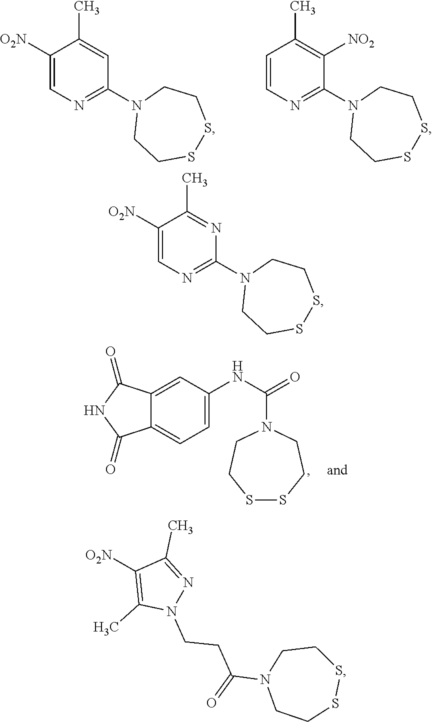
or a pharmaceutically acceptable salt thereof.

14. A compound of formula (III) 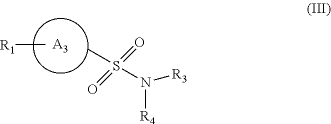
or a pharmaceutically acceptable salt thereof,
wherein
A₃ is a 5- or 6-membered aromatic or heteroaromatic ring comprising at least one heteroatom and at least one carbon atom, wherein A₃ is optionally substituted with one or more R₁ groups, wherein each R₁ is attached to one or more carbon atoms of A₃, and wherein when more than one R₁ is present, then each R₁ may be the same or different,
R₁ is attached to one or more carbon atoms of A₃, and R₁ is independently selected from the group consisting of: hydrogen; (C₁-C₆)alkyl; halo(C₁-C₆)alkyl; (C₁-C₆)alkoxy; halo(C₁-C₆)alkoxy; hydroxy(C₁-C₆)alkyl; (C₁-C₆)alkoxy(C₁-C₆)alkyl; (C₂-C₅)alkenyl; (C₂-C₅)alkynyl; SO₂R₂; (CH₂)₀₋₄CO₂R₂; NH₂; NHR₂; N(R₂)(R₂) wherein each R₂ may be the same or different; (C₁-C₆)alkylamine; di(C₁-C₆)alkylamine; NHCOR₂; hydroxyl; halogen; CN; and NO₂;
wherein R₂ is selected from: hydrogen, (C₁-C₆)alkyl, halo(C₁-C₆)alkyl, hydroxy(C₁-C₆)alkyl, (C₁-C₆)alkoxy(C₁-C₆)alkyl, (C₂-C₅)alkenyl, (C₂-C₅)alkynyl, (C₃-C₆)cycloalkyl, halo(C₃-C₆)cycloalkyl, and hydroxy(C₃-C₆)cycloalkyl; and
R₃ and R₄ are each independently selected from the group consisting of: hydrogen; (C₁-C₁₀) alkyl; halo(C₁-C₆)alkyl; hydroxy(C₁-C₆)alkyl; (C₁-C₆)alkoxy(C₁-C₆)alkyl; (C₂-C₅)alkenyl; (C₂-C₅)alkynyl; (C₃-C₆)cycloalkyl; halo(C₃-C₆)cycloalkyl; hydroxy(C₃-C₆)cycloalkyl; (CHR₅)ₙCO₂R₅;
or R₃ and R₄ together, with the nitrogen to which they are attached to, join together to form a 5-10 membered aromatic or heterocyclic ring or cyclic ring linked to the nitrogen with (CH₂)₁₋₄, optionally substituted at one or more ring atoms with (C₁-C₆)alkyl, halo(C₁-C₆)alkyl, hydroxy(C₁-C₆)alkyl, (C₁-C₆)alkoxy(C₁-C₆)alkyl, hydroxyl, CN, halogen, NH₂; (C₁-C₆)alkylamine; di(C₁-C₆)alkylamine; and NO₂,
wherein R₅ is independently selected from the group consisting of: hydrogen and a (C₁-C₆)alkyl and
wherein n is any number from 0 and 4.

15. The compound of claim 14, wherein at least one R₁ is present, and each R₁ is independently CO₂H, CO₂R₂, OH, or (CH₂)₁₋₄CO₂R₂.

16. The compound of claim 14, wherein at least two R₁ groups are present, and R₁ is CO₂R₂ in one position and hydroxyl at another position.

17. The compound of claim 14, at least one of R3 and R4 is cyclopropane or (C1-C6) alkyl, or R₃ and R₄ join together to form a 5 or 6 membered heterocyclic ring, wherein the heterocyclic ring comprises a further heteroatom that is oxygen or sulfur.

18. The compound of claim 14, wherein A₃ is a benzene ring or thiophene.

19. A compound selected from the group consisting of: 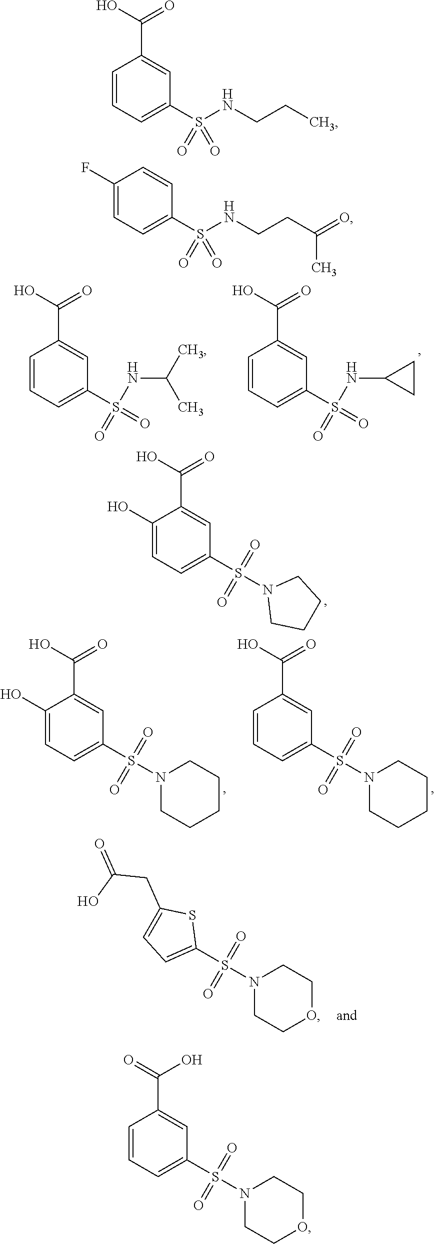
or a pharmaceutically acceptable salt thereof.

20. A compound of formula (IV)
formula (IV): 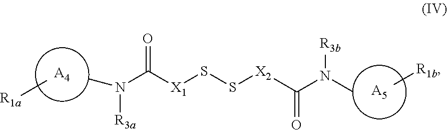
or a pharmaceutically acceptable salt thereof,
wherein
A₄ and A₅ are each independently a 6-14 membered aromatic or heteroaromatic ring comprising at least one heteroatom and at least one carbon atom, wherein each of A₄ and A₅ are independently optionally substituted with R₁ₐ and R_{1b}, respectively,
wherein R₁ₐ and R_{1b} are each independently optionally attached to one or more carbon atoms of the A₄ and A₅, respectively, and R₁ₐ and R_{1b} are each independently selected from the group consisting of: hydrogen; (C₁-C₆)alkyl; halo(C₁-C₆)alkyl; (C₁-C₆)alkoxy; halo(C₁-C₆)alkoxy; hydroxy(C₁-C₆)alkyl; (C₁-C₆)alkoxy(C₁-C₆)alkyl; (C₂-C₅)alkenyl; (C₂-C₅)alkynyl; CO₂R₂; NH₂; NHR₂; N(R₂)(R₂) wherein each R₂ may be the same or different (C₁-C₆)alkylamine; di(C₁-C₆)alkylamine; NHCOR₂; hydroxyl; halogen; CN; and NO₂;
wherein R₂ is selected from the group consisting of: hydrogen, (C₁-C₆)alkyl, halo(C₁-C₆)alkyl, hydroxy(C₁-C₆)alkyl, (C₁-C₆)alkoxy(C₁-C₆)alkyl, (C₃-C₆)cycloalkyl, halo(C₃-C₆)cycloalkyl, and hydroxy(C₃-C₆)cycloalkyl;
each of R₃ₐ and R_{3b} is independently hydrogen or (C₁-C₃)alkyl; and
each of X₁ and X₂ is independently CHR₄, wherein R₄ is hydrogen or (C₁-C₃)alkyl, wherein X₁ and X₂ are the same or different

21. The compound of claim 20, wherein A₄ and A₅ are the same or different and are each independently phenyl or napthyl.

22. The compound of claim 20, wherein one or both of R₁ₐ and R_{1b} is NO₂.

23. The compound of claim 20, wherein at least two R₁ₐ groups, and R₁ₐ is a halogen at one position and (C₁-C₆)alkyl at another position, and at least two R_{1b} groups, and R_{1b} is a halogen at one position and (C₁-C₆)alkyl at another position.

24. The compound of claim 20, wherein X₁ and X₂ are the same, and A₄ with its R₁ₐ substitutions is identical to A₅ with its R_{1b} substitutions.

25. A compound selected from the group consisting of: 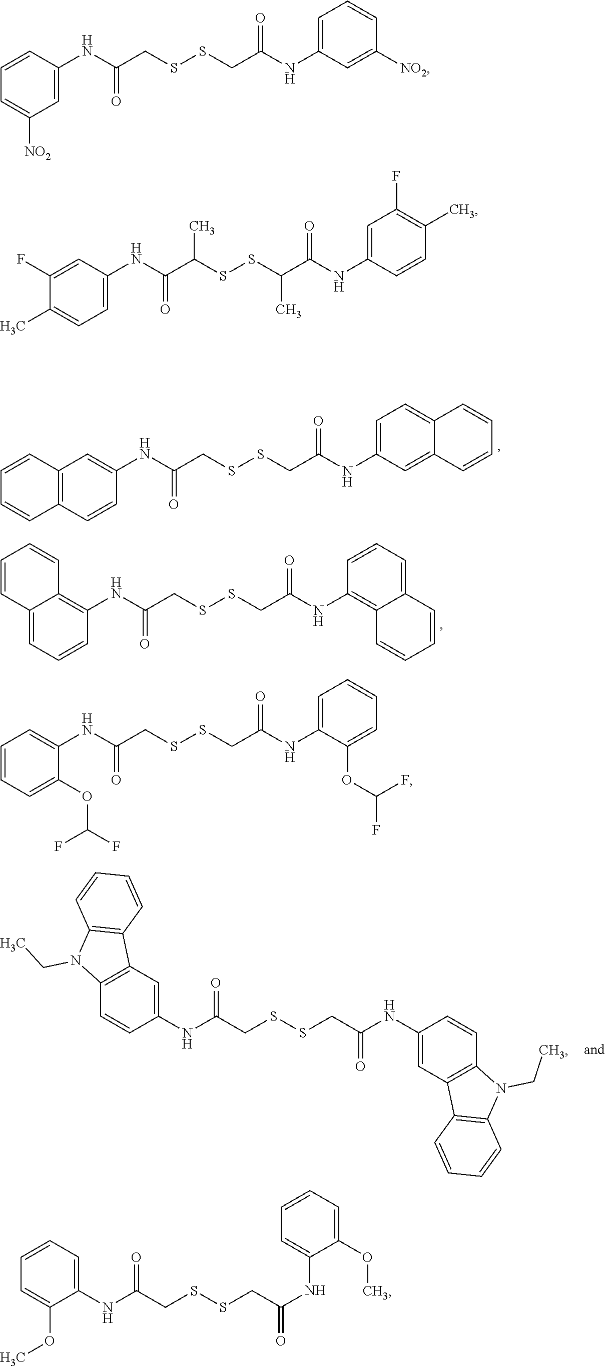
or a pharmaceutically acceptable salt thereof.

26. A compound of formula (V): 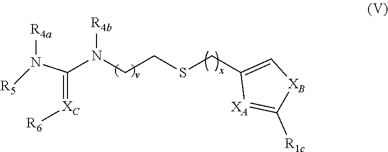
or a pharmaceutically acceptable salt thereof,
wherein:
X_{A} is N or O;
X_{B} is S or CH;
X_{C} is N or CH;
R_{1c} is selected from the group consisting of: (CH₂)₁₋₃NH₂, (CH₂)₁₋₃NH(R₂), (CH₂)₁₋₃NR₂R₃, (CH₂)₁₋₃(C₁-C₆)alkylamine, and (CH₂)₁₋₃di(C₁-C₆)alkylamine, and N═CR₂R₃, wherein R₂ and R₃ are independently hydrogen or (C₁-C₆)alkyl and may be the same or different;
R₄ₐ and R_{4b} are each independently hydrogen or (C₁-C₆)alkyl, and R₄ₐ and R_{4b} may be the same or different,
R₅ is (C₁-C₆)alkyl, (C₂-C₅)alkenyl, (C₂-C₅)alkynyl, or CH₂CN;
R₆ is NH₂, NHR₂, N(R₂)(R₂) wherein each R₂ may be the same or different, NO₂ or (C₁-C₆)alkyl, wherein R₂ is selected from the group consisting of: hydrogen, (C₁-C₆)alkyl, halo(C₁-C₆)alkyl, hydroxy(C₁-C₆)alkyl, (C₁-C₆)alkoxy(C₁-C₆)alkyl, (C₃-C₆)cycloalkyl, halo(C₃-C₆)cycloalkyl, and hydroxy(C₃-C₆)cycloalkyl;
v is any number from 0 to 3; and
x is any number from 0 to 3.

27. The compound of claim 26, wherein when X_{A} is N, then X_{B} is S, or wherein X_{A} is O, then X_{B} is CH.

28. The compound of claim 26, wherein R₅ is CN or (C₁-C₆)alkyl.

29. The compound of claim 26, wherein R₆ is (C₁-C₆)alkyl or NO₂.

30. The compound of claim 26, wherein X_{A} is N; X_{B} is S; v is 2, and x is 1; X_{C} is CH, R₄ₐ is a (C₁-C₃) alkyl; R₅ is hydrogen; R₆ is NO₂, NH₂, NHR₂R₃, or CH₃; and R_{1c} is CHNHR₂ or CHNR₂R₃, wherein R₂ and R₃ are the same or different and are each independently (C₁-C₃)alkyl.

31. The compound of claim 26, wherein X_{A} is O; X_{B} is CH; v is 2, and x is 1; X_{C} is N, R₄ₐ is hydrogen; R₅ is CH₂CN; R₆ is (C₁-C₃)alkyl; and R_{1c} is N═CR₂R₃, wherein R₂ and R₃ are the same or different and are each independently (C₁-C₃)alkyl or hydrogen.

32. A compound selected from the group consisting of: 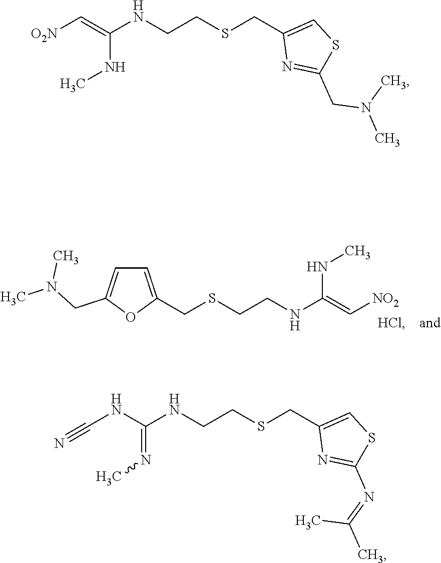
or a pharmaceutically acceptable salt thereof.

33. A pharmaceutical composition comprising a compound of claim 1, and one or more pharmaceutically acceptable excipients.

34. A method of method of treating, preventing, reducing the occurrence of, or reducing, ameliorating, or alleviating the symptoms associated with presbyopia, cataract, or other conditions or disorders associated with the eye, comprising administering an effective amount of a compound of claim 1 to a subject in need thereof.
